(19) United States
(12) Patent Application Publication
(10) Pub. No.: US 2008/0261216 A1
(43) Pub. Date: Oct. 23, 2008

(54) HERV Group II Viruses In Lymphoma And Cancer

(75) Inventors: David Markovitz, Ann Arbor, MI (US); Rafael Contreras-Galindo, Ann Arbor, MI (US); Mark H. Kaplan, Ann Arbor, MI (US); Michael H. Dosik, Setauket, NY (US)

Correspondence Address: Casimir Jones, S.C., 440 Science Drive, Suite 203, Madison, WI, 53711, US

(73) Assignee: The Regents of the University of Michigan, Ann Arbor, MI (US)

(21) Appl. No.: 11/852,369

(22) Filed: Sep. 10, 2007

Related U.S. Application Data
(60) Provisional application No. 60/843,057, filed on Sep. 8, 2006.
(60) Provisional application No. 60/901,484, filed on Feb. 15, 2007.

(51) Int. Cl. C12Q 1/68; G01N 33/574; C12Q 1/02

(52) U.S. Cl. 435/6; 435/7.23; 435/29

(57) ABSTRACT

The present invention relates to compositions and methods for cancer diagnosis and therapy, including but not limited to, cancer markers. In particular, the present invention relates to HERV-K(HML-2) target titers as diagnostic markers, and HERV-K(HML-2) therapeutic targets for HIV-related cancers, and other cancers.

The present invention claims priority to U.S. Provisional Application Ser. No. 60/843,057 filed Sep. 8, 2006, the disclosure of which is hereby incorporated by reference in its entirety, and to U.S. Provisional Application Ser. No. 60/901,484 filed Feb. 15, 2007, the disclosure of which is hereby incorporated by reference in its entirety.

### FIELD OF THE INVENTION

The present invention relates to compositions and methods for cancer diagnosis and therapy, including but not limited to, cancer markers. In particular, the present invention relates to human endogenous retrovirus K HML-2 (HERV-K(HML-2)) target titers as diagnostic markers, and HERV-K(HML-2) therapeutic targets for HIV-related cancers, and other cancers.

### BACKGROUND OF THE INVENTION

HIV-associated lymphoma in the pre highly active antiretroviral therapy (HAART) era occurred in approximately 5-10% of all HIV patients, and were generally large cell lymphomas (LCL) arising in extra nodal areas, for example, in the brain, intestine, lung or other organ sites (Kaplan M H, Susin M, Pahwa S, Fetten J, Allen S L, Lichtman S, Sarngadharan M G, Gallo R C:Neoplastic complications of HTLV III infection: Lymphomas and solid tumors. Amer J Med 82(3):389-396, 1987.). These tumors are aggressive and often show significant necrosis. Since the advent of HAART, their incidence has decreased. (International Collaboration on HIV Infection and Cancer: HAART and incidence of cancer in HIV infected Adults. J Natl Cancer Inst 2000, 92:1823-1830; Besson C, Goubar A, Gabarre J, et al.: Changes in AIDS-related lymphoma since the era of highly active antiretroviral therapy. Blood 2001, 98:2339-2344; Sparano J A. Human Immunodeficiency virus associated lymphoma. Curr Opin Oncol 15:372-6, 2003.). CNS lymphomas of the large cell type have nearly disappeared, but extra-neural large cell lymphoma continues to be of significant risk in patients with poorly controlled viral infection, especially when CD4 counts fall to fewer than 200 cells/mm³. Burkitt's lymphoma (BL) is the second most common lymphoma. These tumors have a characteristic 8/14 c-myc translocation, and generally occur at higher CD4 counts and in the setting of poor HIV viral control. These tumors are aggressive and multicentric, with frequent CNS involvement.

Hodgkin's disease (HD) prior to HAART therapy was unusual, with only a slight increase in incidence in HIV patients. Since the advent of HAART the incidence of this tumor has been increasing. HD arises when CD4 counts are about 200-300 and usually when viral RNA loads are increased. (Levine, A Hodgkin's disease in the setting of human immunodeficiency virus infection. Monogr Natl Cancer Inst. 1998 23:37-42; Cheung T W, Arai S. HIV-associated Hodgkin's disease. AIDS Read. 1999 March-April; 9(2):131-7; Calza L, Manfredi R, Colangeli V, Dentale N, Chiodo F. Hodgkin's disease in the setting of human immunodeficiency virus infection. Scand J Infect Dis. 2003; 35(2):136-41.). In HIV most of these HD tumors are lymphocyte depleted. Disease presents in a more wide spread fashion with “B” symptoms. Almost all CNS lymphomas (Vallat-Decouvelaere A V, Bretel M A, Vassias I, Laplanche J L, Polivka M, Wassef M, Brunet M, Thiebaut J B, Gosselin B, Morinet F, Mikol J. High frequency of a 30-bp deletion of Epstein-Barr virus latent membrane protein 1 gene in primary HIV non-Hodgkin's brain lymphomas. Neuropathol Appl Neurobiol. 2002 December; 28(6):471-9.) and 30-50% of peripheral LCLs and about 20% of BLs are EBV positive. (Knowles, D M. Etiology and pathogenesis of AIDS-related non-Hodgkin's lymphoma. Hematol Oncol Clin North Am. 2003 June; 17(3):785-820. Review. PMID: 12852656.). In HD, the Reed Sternberg cell carries EBV about 40% of the time. EBV is an important contributor to lymphomagenesis and may represent some monoclonal outgrowth of poorly immunologically controlled EBV. However, 50% of large cell lymphomas and most Burkitt's lymphoma and HD arise in the absence of EBV; the cause of these tumors remains elusive.

### SUMMARY OF THE INVENTION

The present invention relates to compositions and methods for cancer diagnosis and therapy, including but not limited to, cancer markers. In particular, the present invention relates to HERV-K(HML-2) target titers as diagnostic markers, and HERV-K(HML-2) therapeutic targets for HIV-related cancers, and other cancers.

In the course of work conducted in the development of the present invention, viral sequences were detected that are associated with HIV-associated lymphomas, non-HIV associated lymphomas and other cancers. Hence, HIV/AIDS-related lymphoma (large cell, Burkitt's and Hodgkin's disease) occurs at increasing frequency in HIV as immunodeficiency progresses and viral load increases. While the present invention is not limited to any particular mechanism and an understanding of the mechanism in not necessary to practice the present invention, it is believed that a virus is responsible for the development AIDS lymphoma, non-HIV associated lymphoma and other cancers. With completion of the Human Genome Project, it is apparent that about 8% of the human genome represents integrated retroviruses most of which are transcriptionally inactive and have multiple mutations and deletions. Many are fragments of older retroviruses. However one group of viruses related to the mouse mammary tumor virus call HERV II K is able to become transcriptionally active. In work conducted in the development of the present invention it was found that a particular group called HML-2 are present in active replicating forms in the plasma of patients with HIV infection, HIV-related cancer, and non-HIV-related cancers. Hence, methods and kits for quantifying HERV-K(HML-2) viruses in the blood of patients are clearly needed.

The present invention is based, in part, on the discovery of HERV-K(HML-2) RNA circulating in the blood of cancer patients. Accordingly, the present invention provides diagnostic, research, and therapeutic methods that target (e.g., detect) the HERV-K(HML-2) (e.g., directly or indirectly). In some embodiments, the present invention provides a method, comprising detecting the presence or absence of HERV-K(HML-2) targets in a sample from a subject, wherein the presence of the HERV-K(HML-2) target is indicative of cancer (e.g., lymphoma, breast cancer) in the subject. For example, in some embodiments, the HERV-K(HML-2) target comprises at least a portion of the HERV-K(HML-2) nucleic acid (e.g. RNA).

Accordingly, in some embodiments, the present invention provides a method of diagnosing cancer in a subject comprising: providing a sample from a subject; contacting said sample with one or more reagents sufficient for detection of an HERV-K(HML-2) target; measuring an amount of said HERV-K(HML-2) target in said sample; and detecting cancer or the risk of cancer in said subject based on said amount of said HERV-K(HML-2) target in said sample. In some embodiments the subject is a human subject. In other embodiments, the cancer is selected from a group consisting of an HIV-related cancer and an HIV-unrelated cancer. In further embodiments the HIV-related cancer is selected from a group consisting of HIV/AIDS positive large cell lymphoma, HIV/AIDS positive central nervous system lymphoma, HIV positive Hodgkin's disease, and HIV positive T cell leukemia. In still further embodiments, the HIV-unrelated cancer is selected from the group consisting of HIV negative large cell lymphoma, HIV negative Hodgkin's disease, and chronic lymphocytic leukemia. In yet further embodiments the HIV-unrelated cancer is breast cancer.

In some embodiments of the present invention, the sample is selected from, for example, a group consisting of a blood sample, a blood derivative sample, a serum sample, a plasma sample, an effusion, a tissue biopsy, a blood product to be transfused, or an organ or other tissue to be transplanted. In other embodiments, HERV-K(HML-2) target is a nucleic acid. In preferred embodiments, the HERV-K(HML-2) nucleic acid target is RNA. In yet other embodiments the HERV-K(HML-2) target nucleic acid is gag nucleic acid. In further embodiments the HERV-K(HML-2) target nucleic acid is env nucleic acid. In particularly preferred embodiments, HERV-K(HML-2) target nucleic acid is both gag and env nucleic acid, that are, for example, detected sequentially or serially. In additional embodiments, the pattern of HERV-K(HML-2) env subtype target nucleic acids that are detected in a sample from a subject corresponds to the diagnosis of a specific HIV-related or HIV-unrelated cancer in the subject. In some embodiments, the pattern of gag and env genotypes present in a sample from a subject correspond to, for example, the diagnosis of cancer, the type of cancer, the aggressiveness of cancer, the metastatic potential of cancer, the response to therapy of a cancer, the resistance to therapy of a cancer, and the likelihood of a cancer to recur. In some embodiments, the pattern of gag and env genotypes present in a sample from a subject correspond to the presence of one or more subtypes of HERV-K(HML-2) virions in a sample. In a preferred embodiment, the pattern of gag and env genotypes present in a sample from a subject correspond to the presence of one or more replicating HERV-K(HML-2) virions in a sample. In another embodiment, the pattern of gag and env genotypes present in a sample from a subject correspond to the presence of one or more recombinant subtypes of HERV-K(HML-2) virions in a sample.

In a particularly preferred embodiment, the measuring of the amount of the HERV-K (HML-2) target uses nucleic acid sequence based amplification (NASBA). In some embodiments the HERV-K(HML-2) target is HERV-K(HML-2) RNA and the amount of the target is equal to or greater than 10³ copies of HERV-K(HML-2) RNA/mL.

In some embodiments of the present invention, the detection of cancer or the risk of cancer in a subject comprises detecting a response to therapy. In other embodiments the HERV-K(HML-2) target is HERV-K(HML-2) RNA and the amount of the target is equal to or less than 10³ copies of HERV-K(HML-2) RNA/mL in detecting a response to therapy. In further embodiments the detecting is detecting a decrease of HERV-K(HML-2) RNA copies/mL after therapy. In other embodiments, the HERV-K(HML-2) target is a polypeptide.

In some embodiments, the present invention provides a method for screening compounds, comprising: providing: a sample from a subject suspected of having cancer; one or more reagents sufficient for the detection of an HERV-K(HML-2) target; and one or more test compounds; and contacting the biological sample with the one or more test compounds; and detecting an amount of the HERV-K(HML-2) target in the sample using the reagents. In some embodiments the test compound decreases the amount of said HERV-K(HML-2) target in the biological sample. In other embodiments, the test compound increases the amount of said HERV-K(HML-2) target in the biological sample. In a further embodiment, the test compound is a small molecule. In another embodiment, the compound is an antibody. In yet another embodiment, the test compound inhibits the interaction of an HERV-K(HML-2) target with a second compound. In still another embodiment, the sample is an in vitro sample. In an additional embodiment, the said sample is an in vivo sample. In a preferred embodiment, the test compound treats cancer in a subject.

In some embodiments, the present invention provides a kit for diagnosing cancer in a subject, comprising one or more reagents sufficient for detection of an HERV-K(HML-2) target in a sample; and a computer program on a computer readable medium comprising instructions which direct a processor to analyze data derived from use of said reagents to indicate the presence or absence of cancer in a subject. In some embodiments the one or more reagents sufficient for detection of an HERV-K(HML-2) target are reagents configured for nucleic acid sequence based amplification (NASBA).

In another embodiment, the present invention provides a kit to determine the sensitivity of cancer cells to an agent or combination of agents selectively targeting HERV-K(HML-2), comprising: a cancer cell preparation; an agent or combination of agents selectively targeting HERV-K(HML-2); and one or more reagents sufficient to perform an assay selected from the group comprising an assay of cell growth or survival under specific culture conditions, an assay of the ability to express a specific biologic factor, an assay of cell structure, or an assay of differential gene expression.

In some embodiments, HERV-K(HML-2) targets are detected at the level of nucleic acid (e.g., DNA or RNA). In other embodiments, protein polypeptides are detected. In some embodiments, the protein produced contains amino acid sequences encoded by HERV-K(HML-2) RNA. In some such embodiments, the protein or peptide produced differs in sequence, post-translational processing, and/or structure from the associated natural protein and the difference is detected to identify the presence of the HERV-K(HML-2) RNA.

The present invention is not limited by the nature of the sample that is tested for the presence of the HERV-K(HML-2) target. In some embodiments, the sample is tissue (e.g., biopsy), blood, urine, circulating cells, or semen, or a component thereof. Serum is particularly useful for non-invasive methods of the present invention.

In some embodiments, the sample comprises a biopsy sample (e.g., a lymphoma or breast biopsy sample). In some embodiments, the sample comprises a urine sample or a component of a urine sample.

In some embodiments, the detecting the presence or absence of HERV-K(HML-2) target comprises detection of a nucleic acid molecule (e.g., via polymerase chain reaction (PCR) or quantitative PCR, reverse transcriptase PCR, ligase-mediated rapid amplification of cDNA ends, microarray analysis, transcription-mediated amplification (TMA), nucleic acid sequence-based amplification (NASBA) analysis (for example, bioMerieux, Marcy l'Etoile, France), ligase chain reaction (LCR), strand displacement amplification (SDA), loop-mediated amplification, sequencing, etc.). In other embodiments, the detection method comprises detecting HERV-K(HML-2) target in a tissue sample (e.g., using fluorescence in situ hybridization (FISH)).

In some embodiments, the method further comprises the step of diagnosing or detecting cancer in the subject based on the presence or absence HERV-K(HML-2) target above threshold levels of viral load. In some embodiments, the presence of HERV-K(HML-2) target is indicative of the presence of cancer in the subject. In some embodiments, the presence of, nature of, or amount of expression of HERV-K(HML-2) target is indicative of the nature of the cancer (e.g., type of cancer, progression of cancer, stage of cancer, risk of metastasis, presence of metastasis, etc.).

In still other embodiments, the present invention provides a kit comprising reagents for detecting (e.g., sufficient for detecting) the presence or absence of HERV-K(HML-2) target in a sample. Kit components include, but are not limited to, hybridization oligonucleotides or polynucleotides (e.g., probes, primers, FISH probes, etc.), enzymes (e.g., polymerases, ligases, reverse transciptases, nucleases, etc.), buffers, containers for housing components, filters, sample isolation and preparation components, software, instrumentation, and the like. In some embodiments, the kit further comprises instructions (e.g., written instructions, software, instructions on computer readable media, etc.) for detecting or diagnosing cancer in the subject based on the presence or absence of HERV-K(HML-2) targets. In some embodiments the instructions further provide a recommended course of action based on the results of the analysis (e.g., to assist a treating physician in optimizing care for a patient).

Additional embodiments of the present invention are described in the description and examples below.

### DESCRIPTION OF THE FIGURES

FIG. 1 shows a phylogenetic dendogram of 244 bp HERV-K pol sequences amplified from HIV-1 patients (black circles), together with reported HERV-K subfamilies (HLM1 to HLM10) and type A, B, C and D retrovirus.

FIG. 2 shows amplification of HERV-K viral RNA from HIV-1+ plasma samples. FIG. 2A. shows the genomic organization of HERV-K viral RNA of type-1 and type-2 viruses. HERV-K type-1 lacks a 292 bp nucleotide boundary (▴) that fuses the viral genes pol and env. The 292 bp segment in type-2 viruses has nucleotide sequences that code for the first exon of rec. On the other hand, type-1 HERV-K viruses code for the accessory protein, np9, whose viral function is unknown. In the illustrations between the HERV-K genomes are the primers used: they are located in perspective to the regions they anneal. FIG. 2B shows amplification of HERV-K genes in HIV-1 patients. Shown are the amplifications of gag, prt, pol, env, and the U5-pol segment representing (a) the six HIV-1+ patients, (b) the six HIV-1+/HCV+ patients, (c) the six HCV+ patients, (d) the six healthy volunteers, and (e) the negative controls: dH₂O. L1: Biomarker low (Bioventures, Inc.), L2: 1 Kb Ladder (Promega). As depicted in the figure of env SU amplification, the lower band represents type-1 viruses (˜1100 bp) and the upper band represents type-2 viruses (˜1392 bp).

FIG. 3 shows HERV-K RNA titers in plasma from control subjects, HIV-1 positive, AIDS related lymphomas and other cancers. HERV-K RNA titers were measured by Real Time RT-PCR. The scatter box blot represents the log₁₀ HERV-K RNA values in each patient. Patients are grouped by disease. Lines indicate the log HERV-K(HML-2) RNA mean.

FIG. 4 shows HERV-K RNA titers in plasma from lymphoma patients during disease onset and remission. HERV-K RNA titers were measured by Real Time RT-PCR. The scatter box blot represents the log₁₀ HERV-K RNA values in each patient. Patients are grouped by disease. Lines indicate the log HERV-K(HML-2) RNA mean.

FIG. 5 shows a computerized axial tomography scan showing the appearance of the right (upper) and left (lower) kidney from a Large cell lymphoma patient with CMV retinitis at the time of the diagnosis (A) and after treatment with PFA (B). The large cell lymphoma is observed on the right kidney (arrow).

FIG. 6 shows the reduction in the HERV-K viral load to an undetectable level after the start of foscarnet. This was accompanied by a spontaneous regression of the patients large cell lymphoma of the kidney as shown in FIG. 5.

FIG. 7 shows that HERV-K(HML-2) RNA titers are reduced in a patient receiving PFA. An increase in HERV-K(HML-2) RNA titers is observed after PFA therapy is interrupted. HIV RNA titers are not affected by PFA. (HIVVL: squares, HERV-K(HML-2) viral burden: circles).

FIG. 8 shows that HERV-K(HML-2) RNA titers are suppressed in a second patient receiving with CMV retinitis and CNS lymphoma PFA. An increase in HERV-K(HML-2) RNA titers is observed after PFA therapy is interrupted. HIV RNA titers are not affected by PFA. (HIVVL: squares, HERV-K(HML-2) viral burden: circles).

FIG. 9 shows recombination plots of recombination plots of HERV-K(HML-2) env sequences from the K151 breast cancer cell line.

FIG. 10 shows a phylogenetic neighbor-joining tree of type-1 HERV-K(HML-2) env (SU) sequences amplified from breast cancer patients, and from the cell line K151.

FIG. 11 shows HERV K env DNA fragments obtained from Hodgkin's disease patients by RT PCR from RNA in plasma-derived templates.

FIG. 12 shows HERV-K RNA titers, reverse transcriptase (RT) activity, and Western blots from sucrose gradient fractions from plasma samples of two lymphoma patients (Patient 1, top, and Patient 2, bottom). The hollow bars show HERV-K RNA titers and the solid bars show RT activity.

FIG. 13 shows Western blotting of 30% iodoxinol cushions from plasma samples of lymphoma patients. Lane A shows cell lysate of HERV-K-particle negative cell line PA-1. Lanes B, C and D show plasma samples from Large Cell Lymphoma patients with high HERV-K RNA titers.

FIG. 14 shows a phylogenetic neighbor-joining (NJ) tree of Type-1 HERV-K (HML-2) env SU sequences amplified from the plasma of patients with Hodgkin's Disease.

FIG. 15 shows a phylogenetic neighbor-joining (NJ) tree of Type-1 and Type-2 HERV-K (HML-2) env SU sequences amplified from the plasma of patients with Large Cell Lymphoma (LCL).

FIG. 16 shows a phylogenetic neighbor-joining (NJ) tree of Type-1 HERV-K (HML-2) env SU sequences amplified from the plasma of patients with breast cancer.

### GENERAL DESCRIPTION

Approximately 8 percent of the human genome sequence is composed by human endogenous retroviruses (HERVs), most of which are replication defective. HERV-K(HML-2) is phylogenetically the youngest and most active family, and has maintained some proviruses with intact open reading frames (ORFs) which code for viral proteins that may assemble into viral particles. Many HERV-K(HML-2) sequences are polymorphic in humans (i.e., specific variants are present in some individuals but not in others), and others may be unfixed (i.e., not inserted permanently in a specific chromosomal location of the human genome). Patients with advanced AIDS are at progressive risk of developing large cell lymphoma (LCL), Burkitt's lymphoma (BL) or Hodgkin's disease (HD). Forty percent of these tumors are associated with the Epstein Barr virus (EBV) but no other viral entity has been identified in the remaining 60%, suggesting that another retrovirus causes these complications of lymphoma.

In work conducted in the course of development of the present invention it was discovered that when a patient is infected with HIV the HERV viruses become active. A group of viruses called HML-2 (subdivided into types 1 and 2) that are related to the mouse mammary tumor virus are present in high titers in the plasma of patients with HIV-associated lymphomas. There are approximately 10 distinct subtypes of these viruses in the human genome that may undergo activation. In work conducted in the course of development of the present invention a quantitative assay was developed for the envelope gene of HML-2. Patients with HIV-associated lymphoma exhibit elevated titers of the HERV-K targets in their plasma (for example, >100,000,0000 copies of gag and/or env). These titers reach their peak at the peak of lymphoma and clear from the plasma with treatment of lymphoma.

### DEFINITIONS

To facilitate an understanding of the present invention, a number of terms and phrases are defined below:

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

In some embodiments of the present inventions a subject is selected from a group consisting of subject at risk for developing cancer, a subject suspected of having cancer, a subject suspected of having cancer metastasis, a subject suspected of having cancer recurrence, a subject known to have cancer, a subject undergoing cancer therapy, and a subject that has completed cancer therapy.

As used herein, the term “subject suspected of having cancer” refers to a subject that presents one or more symptoms indicative of a cancer) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass or increased PSA level, breast cancer or lymphoma biopsy, leukemic cells in the circulation or marros), but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “characterizing cancer tissue in a subject” refers to the identification of one or more properties of a cancer tissue sample (e.g., including but not limited to, the presence of cancerous tissue, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “cancer marker genes” refers to a gene or genes whose presence or expression level, alone or in combination with other genes, is correlated with cancer or prognosis of cancer. The correlation may relate to either an increased or decreased expression of the gene. For example, the expression of the gene may be indicative of cancer, or lack of expression of the gene may be correlated with poor prognosis in a cancer patient.

As used herein, the term “a reagent that specifically detects the presence or absence of HERV-K(HML-2) target” refers to reagents used to detect the presence of or expression of one or more HERV-K(HML-2) targets (e.g., including but not limited to, the cancer markers of the present invention). Examples of suitable reagents include but are not limited to, nucleic acid probes capable of specifically hybridizing to the HERV-K(HML-2) targets of interest, PCR primers capable of specifically amplifying the gene of interest, and antibodies capable of specifically binding to proteins expressed by the gene of interest. Other non-limiting examples can be found in the description and examples below.

As used herein, the term “instructions for using said kit for detecting cancer in said subject” includes instructions for using the reagents contained in the kit for the detection and characterization of cancer in a sample from a subject. In some embodiments, the instructions further comprise the statement of intended use required by the U.S. Food and Drug Administration (FDA) in labeling in vitro diagnostic products.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “initial diagnosis” refers to results of initial cancer diagnosis (e.g. the presence or absence of cancerous cells). An initial diagnosis does not include information about the stage of the cancer of the risk.

As used herein, the term “biopsy tissue” refers to a sample of tissue (e.g., breast or lymph node tissue) that is removed from a subject for the purpose of determining if the sample contains cancerous tissue. In some embodiment, biopsy tissue is obtained because a subject is suspected of having cancer. The biopsy tissue is then examined (e.g., by microscopy) for the presence or absence of cancer.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum, and non-blood products, for example, urine, spinal fluid, bile, saliva, stool, tears, sweat, mucous, semen, cells, and tissues, and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates to compositions and methods for cancer diagnosis and therapy, including but not limited to, cancer markers. In particular, the present invention relates to HERV-K(HML-2) target titers as diagnostic markers, and HERV-K(HML-2) therapeutic targets for HIV-related cancers, and other cancers. Accordingly, the present invention provides methods and kits for the detection of markers, as well as drug screening and therapeutic applications.

The human genome harbors numerous retroviral sequences that comprise up to 8% of the host genome, many of which have accumulated lethal mutations that have impaired their ability to replicate. (Nelson P, Carnegie P, Martin J, Davari E, Hooley P, Roden D, Rowland-Jones S, Warren P, Astley J and Murray P: Demystified Human endogenous retroviruses. Mol Pathol 2003; 56:11-18; Wang-Johanning F, Frost A, Jian B, Epp L, Lu D and Johanning G: Quantitation of HERV-K env gene expression and splicing in human breast cancer. Oncogene 2003; 22:1528-1535; Hughes J and Coffin, J: Human endogenous retrovirus K solo-LTR formation and insertional polymorphisms: implications for human and viral evolution. Proc Natl Acad Sci USA 2004; 101: 1688-1672). The human endogenous retrovirus type-K (HERV-K.HML-2) family is represented by many proviruses, some of which possess intact open reading frames (ORFs) for gag, prt, pol, and env genes. (Barbulescu M, Turner G, Seaman M, Deinard A, Kidd K and Lenz J: Many human endogenous retrovirus K (HERV-K) proviruses are unique to humans. Curr Biol 1999; 9:861-868; Paces J, Pavlicek A and Paces V: HERVd: the Human Endogenous Retroviruses Database. Nucleic Acids Res 2002; 30:205-206). To date, HERV-K(HML-2) is the only endogenous retroviral subfamily with the ability to produce viral particles. (Bannert N and Kurth R: Retroelements and the human genome: new perspectives on an old relation. Proc Natl Acad Sci USA 2002;101 Suppl 2:14572-14579; Simpson G, Patience C, Lower R, Tonjes R, Moore H, Weiss R and Boyd M: Endogenous D-type (HERV-K) related sequences are packaged into retroviral particles in the placenta and possess open reading frames for reverse transcriptase. Virology 1996; 222:451-456; Bieda K, Hoffmann A and Boller K: Phenotypic heterogeneity of human endogenous retrovirus particles produced by teratocarcinoma cell lines. J Gen Virol 2001; 3:591-596; Boller K, Konig H, Sauter M, Mueller-Lantzsch N, Lower R, Lower J and Kurth R: Evidence that HERV-K is the endogenous retrovirus sequence that codes for the human teratocarcinoma-derived retrovirus HTDV. Virology 1993;1:349-353). However, an intact HERV-K proviral sequence (K113) and perhaps other unidentified unfixed elements may code for replication-competent viruses. (Turner G, Barbulescu M, Su M, Jensen-Seaman M, Kidd K and Lenz J: Insertional polymorphisms of full-length endogenous retroviruses in humans. Curr Biol 2001; 11:1531-1535; Moyes D, Martin A, Sawcer S, Temperton N, Worthington J, Griffiths D and Venables P: The distribution of the endogenous retroviruses HERV-K113 and HERV-K115 in health and disease. Genomics 2005; 86:337-341; Bleshaw R, Dawson A L, Woolven-Allen J, Redding J, Burt A, Tristem M. Genomewide screening reveals high levels of insertional polymorphism in the human endogenous retrovirus family HERV-K(HML2): implications for present-day activity. J Virol 2005;79: 12507-12514). Lower et. al reported the detection of anti-HERV-K antibodies in the plasma of 70% of HIV-1 patients compared to only 3% of healthy blood donors. (Lower R, Lower J and Kurth R: The viruses in all of us: characteristics and biological significance of human endogenous retrovirus sequences. Proc Natl Acad Sci USA 1996; 93:5177-5184). Antibodies to HERV-K were also detectable in drug users, but only after HIV-1 seroconversion. (Vogetseder W, Dumfahrt A, Mayersbach P, Schonitzer D and Dierich M: Antibodies in human sera recognizing a recombinant outer membrane protein encoded by the envelope gene of the human endogenous retrovirus K. AIDS Res Hum Retroviruses 1993; 9:687-694). In work conducted in the course of development of the present invention, it was found that if HERV-K viral particles are made, they may be protected by viral envelopes in plasma of HIV-1 infected individuals, and that the RNA genome is directly amplified from viral RNA extractions of plasma.

### I. Markers for Cancer

The present invention provides markers that are specifically altered in cancerous tissues (e.g. in breast, lymph node and bone marrow tissue). Such markers find use in the diagnosis and characterization of cancer. In some embodiments, the present invention.

The present invention is not limited to a particular HERV-K(HML-2) target sequence. Exemplary HERV-K(HML-2) target sequences are described below.

### II. Diagnostic Applications

In some embodiments, the present invention provides methods for detection of the existence of or expression of cancer markers (e.g., HERV-K(HML-2) targets). In the present invention HERV-K(HML-2) targets are detected. In some embodiments, the presence of HERV-K(HML-2) target is confirmed (e.g., using a hybridization assay) and the size of HERV-K(HML-2) targets confirms the presence of HERV-K(HML-2) targets. In some embodiments, a protein or other gene expression product is detected. In some embodiments, the form (e.g., amino acid sequence, folding, size, shape, post-translational processing, location in a cell, association with other proteins, etc.) of the protein or other gene expression product generated by the HERV-K(HML-2) target differs from a native protein.

In some embodiments, an initial assay confirms the presence of a HERV-K(HML-2) target but does not identify the specific HERV-K(HML-2) target. For example, in some embodiments, multiplex assays are utilized where a positive result is indicative of the presence of HERV-K(HML-2) targets. A secondary assay is then performed to determine the identity of the HERV-K(HML-2) target, if desired. In some embodiments, the second assay uses a different detection technology than the initial assay. In certain embodiments, the second assay utilizes DNA sequencing methods.

In some embodiments, expression is measured directly (e.g., at the DNA, RNA or protein level). The diagnostic methods of the present invention are suitable for the detection of any of the possible HERV-K(HML-2) targets, transcripts, or proteins.

In some embodiments, the presence of HERV-K(HML-2) targets or expression from HERV-K(HML-2) targets is detected in tissue samples (e.g., biopsy tissue). In other embodiments, HERV-K(HML-2) target is detected in bodily fluids (e.g., including but not limited to, plasma, serum, circulating cells, whole blood, mucus, saliva, and urine). The methods of the present invention are suitable for detection of amplified or unamplified nucleic acid samples.

In some embodiments, the presence of a cancer marker is used to provide a prognosis to a subject. For example, the detection of HERV-K(HML-2) target is indicative of breast cancer. The information provided is also used to direct the course of treatment. For example, if a subject is found to have a marker indicative of a highly metastasizing tumor, additional therapies (e.g., hormonal, surgical or radiation therapies) can be started at an earlier point when they are more likely to be effective (e.g., before metastasis). In addition, if a subject is found to have a tumor that is not responsive to hormonal therapy, the expense and inconvenience of such therapies can be avoided. Conversely, if a subject is found to have a marker indicative of a less aggressive tumor or is identified at risk for developing cancer, a watchful waiting program can be instituted. In some embodiments, the presence or absence of a particular HERV-K(HML-2) target (e.g., in a blood or urine sample) is utilized to determine if a biopsy is necessary. For example, in some embodiments, the absence of the marker or the detection of a HERV-K(HML-2) target is indicative of a less aggressive form of cancer can be used to determine that a patient can be spared an unpleasant and invasive biopsy.

In certain embodiments, the HERV-K(HML-2) target of the present invention is identified in combination with another marker for cancer. In some embodiments, the marker includes, but is not limited to, a radiologic image (for example, a CT scan), or a second blood antigen (for example, PSA or CEA).

In some embodiments, the present invention provides a panel for the analysis of a plurality of markers. The panel allows for the simultaneous analysis of multiple markers correlating with carcinogenesis and/or metastasis. For example, a panel may include markers identified as correlating with cancerous tissue, metastatic cancer, localized cancer that is likely to metastasize, pre-cancerous tissue that is likely to become cancerous, and pre-cancerous tissue that is not likely to become cancerous. Depending on the subject, panels may be analyzed alone or in combination in order to provide the best possible diagnosis and prognosis. Markers for inclusion on a panel are selected by screening for their predictive value using any suitable method, including but not limited to, those described in the illustrative examples below. Panels may also include markers useful in diagnosing other types of cancer or other diseases, infections, metabolic conditions, or other desired aspects of the subject or the subject's environment.

In a preferred embodiment, the present invention provides a method of screening blood before transfusion for HERV-K(HML-2) targets that detect the presence of replicating or transferable agents.

### A. Detection of RNA

In some preferred embodiments, detection of HERV-K(HML-2) target markers (e.g., including but not limited to, those disclosed herein) is detected by measuring the presence of corresponding mRNA in a tissue or blood sample. mRNA may be measured by any suitable method, including but not limited to, those disclosed below.

In some embodiments, RNA is detected by Northern blot analysis. Northern blot analysis involves the separation of RNA and hybridization of a complementary labeled probe.

In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to an oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR(RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In some preferred embodiments, transcription mediated amplification (Gen-Probe, San Diego, Calif.) is utilized for the detection of RNA or DNA (See e.g., U.S. Pat. Nos. 5,399,491 and 5,554,516, each of which is herein incorporated by reference in its entirety). TMA is an RNA transcription amplification system using two enzymes to drive the reaction: RNA polymerase and reverse transcriptase. TMA is isothermal; the entire reaction is performed at the same temperature in a water bath or heat block. This is in contrast to other amplification reactions such as PCR or LCR that require a thermal cycler instrument to rapidly change the temperature to drive the reaction.

TMA can amplify either DNA or RNA, and produces RNA amplicon, in contrast to most other nucleic acid amplification methods that only produce DNA. TMA has very rapid kinetics resulting in a billion-fold amplification within 15-30 minutes. In some embodiments, TMA is combined with a hybridization based detection method (e.g., GEN-PROBE Hybridization Protection Assay (HPA)) in a single tube format. There are no wash steps, and no amplicon is ever transferred out of the tube, which simplifies the procedure and reduces the potential of contamination.

In particularly preferred embodiments, RNA is detected by nucleic acid sequenced based analysis (for example, NASBA (bioMerieux, Marcy l'Etoile, France). NASBA is an isothermal, enzyme-based method for the amplification of nucleic acid. In preferred embodiments the NASBA assay is more sensitive than RT-PCR methods, and is able to directly amplify viral RNA and not DNA. See, for example: U.S. Pat. No. 5,130,238 to Malek, entitled “Enhanced nucleic acid amplification process”; U.S. Pat. No. 6,300,068 entitled “Nucleic acid assays”, EP Patent No.: EP-A-0 329 822; and L. Malek et al., “Nucleic Acid Sequence-Based Amplification (NASBA.TM.)”, Ch. 36 in Methods in Molecular Biology, Vol. 28: 253-260, Protocols for Nucleic Acid Analysis by Nonradioactive Probes, 1994 Ed. P. G. Isaac, Humana Press, Inc., Totowa, N.J., each of which is hereby incorporated by reference in its entirety. Thus, quantification of specific HERV subtypes (for example, subtype 1 and subtype 2) by NASBA may add diagnostic and prognostic information in cancer etiologies to that available from other methods. In some embodiments, NASBA uses a mixture of reverse transcriptase, ribonuclease-H, RNA polymerase, and transcript-specific DNA primers. In other embodiments, one or more NASBA primers comprise a T7 or other priming sites. For example, in some embodiments, a first primer comprises a 5′ extension containing the promoter sequence for bacteriophage T7 DNA-dependent RNA polymerase, and a second primer comprises a 5′ extension containing a complementary binding sequence for an electro-chemiluminescent (ECL) tag. During amplification, the 5′ primer extensions are incorporated into the amplified sequence allowing efficient production of a specific RNA template. The technique is particularly suited for the amplification of single stranded RNA. With optimum conditions a 10¹²-fold level of amplification is possible.

In some embodiments, after amplification, detection may be performed by an additional capture probe, which confirms the presence of RNA amplicon of interest. In preferred embodiments, an aliquot of the amplification reaction is added to a hybridization solution containing both the capture probe and a detection probe. The capture probe is specific for the RNA amplicon of interest, while the detection probe is generic and has complementary region to the RNA amplicon. In further embodiments, the probes comprise complementary ends for quenching fluorophores, for example, FAM or ROX. After incubation, magnetic beads carrying the hybridized amplicon/detection probe complexes may be magnetically captured on the surface of an electrode. Voltage applied to this electrode triggers the detection reaction. Light emitted by the hybridized ruthenium-labelled probe is proportional to the amount of amplicon generated in the corresponding amplification reaction. Detection may also be carried out in a microtiter plate.

### B. Detection of DNA

In other embodiments, HERV-K(HML-2) target (e.g., cDNA) is detected. DNA may be detected using any suitable method. For example, in some embodiments, DNA is detected in vitro (e.g., using nucleic acid probes).

### 1. Direct Sequencing Assays

In some embodiments of the present invention, HERV-K(HML-2) target sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest is sequenced using any suitable method, including but not limited to manual sequencing using radioactive marker nucleotides, or automated sequencing. The results of the sequencing are displayed using any suitable method. The sequence is examined and the presence or absence of a given sequence is determined.

### 2. PCR Assay

In some embodiments of the present invention, HERV-K(HML-2) target sequences are detected using a PCR-based assay. In some embodiments, the PCR assay comprises the use of oligonucleotide primers that hybridize only to the wild type of HERV-K(HML-2) target gene. Both sets of primers are used to amplify a sample of DNA. If only the variant HERV-K(HML-2) target primers result in a PCR product, then the patient has the HERV-K(HML-2) target. If only the wild-type primers result in a PCR product, then the patient has the wild type HERV-K(HML-2) target.

3. Mutational Detection by dHPLC

In some embodiments of the present invention, HERV-K(HML-2) target sequences are detected using a PCR-based assay with consecutive detection of nucleotide variants by dHPLC (denaturing high performance liquid chromatography). Exemplary systems and methods for dHPLC include, but are not limited to, WAVE (Transgenomic, Inc; Omaha, Nebr.) or VARIAN equipment (Palo Alto, Calif.).

### 4. RFLP Assay

In some embodiments of the present invention, HERV-K(HML-2) target sequences are detected using a restriction fragment length polymorphism assay (RFLP). The region of interest is first isolated using PCR. The PCR products are then cleaved with restriction enzymes known to give a unique length fragment for a given HERV-K(HML-2) target. The restriction-enzyme digested PCR products are separated by agarose gel electrophoresis and visualized by ethidium bromide staining. The length of the fragments is compared to molecular weight markers and fragments generated from wild-type and variant HERV-K(HML-2) target controls.

### 5. Hybridization Assays

In preferred embodiments of the present invention, HERV-K(HML-2) target sequences are detected a hybridization assay. In a hybridization assay, the presence of absence of a given HERV-K(HML-2) target is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

a. Direct Detection of Hybridization

In some embodiments, hybridization of a probe to the sequence of interest (e.g., a HERV-K(HML-2) target) is detected directly by visualizing a bound probe (e.g., a Northern or Southern assay; See e.g., Ausabel et al. (eds.), Current Protocols in Molecular Biology, John Wiley & Sons, NY [1991]). In a these assays, cDNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the variant or wild-type HERV-K(HML-2) target is allowed to contact the membrane under a condition or low, medium, or high stringency conditions. Unbound probe is removed and the presence of binding is detected by visualizing the labeled probe.

b. Detection of Hybridization Using “DNA Chip” Assays

In some embodiments of the present invention, HERV-K(HML-2) target sequences are detected using a DNA chip hybridization assay. In this assay, a series of oligonucleotide probes are affixed to a solid support. The oligonucleotide probes are designed to be unique to a given variant or wild-type HERV-K(HML-2) target. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given gene HERV-K RNA are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding,

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the HERV-K(HML-2) target of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of HERV-K(HML-2) target (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given HERV-K(HML-2) target. Batches of beads are combined to form a pool specific to the array. To perform an assay, the BEAD ARRAY is contacted with a prepared subject sample (e.g., DNA or RNA). Hybridization is detected using any suitable method.

c. Enzymatic Detection of Hybridization

In some embodiments of the present invention, hybridization is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific sequences in unamplified cDNA. The isolated cDNA sample is contacted with the first probe specific either for a variant or wild-type HERV-K(HML-2) target sequence and allowed to hybridize. Then a secondary probe, specific to the first probe, and containing the fluorescein label, is hybridized and the enzyme is added. Binding is detected by using a fluorescent plate reader and comparing the signal of the test sample to known positive and negative controls.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, HERV-K(HML-2) targets are detected using the SNP-IT primer extension assay (Orchid Biosciences, Princeton, N.J.; See e.g., U.S. Pat. Nos. 5,952,174 and 5,919,626, each of which is herein incorporated by reference). In this assay, HERV-K RNA are identified by using a specially synthesized DNA primer and a DNA polymerase to selectively extend the DNA chain by one base at the suspected HERV-K RNA location. cDNA in the region of interest is amplified and denatured. Polymerase reactions are then performed using miniaturized systems called microfluidics. Detection is accomplished by adding a label to the nucleotide suspected of being at the HERV-K RNA location. Incorporation of the label into the DNA can be detected by any suitable method (e.g., if the nucleotide contains a biotin label, detection is via a fluorescently labeled antibody specific for biotin).

### 6. Mass Spectroscopy Assay

In some embodiments, a MassARRAY system (Sequenom, San Diego, Calif.) is used to detect HERV-K(HML-2) targets (See e.g., U.S. Pat. Nos. 6,043,031; 5,777,324; and 5,605,798; each of which is herein incorporated by reference). RNA or (cDNA from RNA) is isolated from blood samples using standard procedures. Next, specific DNA regions containing the region of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization—Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

### C. Detection of Protein

In other embodiments, HERV-K(HML-2) cancer markers are detected by measuring the expression of the corresponding protein or polypeptide. Protein expression may be detected by any suitable method. In some embodiments, proteins are detected by immunohistochemistry. In other embodiments, proteins are detected by their binding to an antibody raised against the protein. The generation of antibodies is described below. In some embodiments, antibodies are generated that recognize altered three-dimensional structures in a HERV-K(HML-2) target or protein generated from a HERV-K(HML-2) transcript (e.g., due to truncations or altered structure) but not the wild type protein.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference.

In still further embodiments, HERV-K(HML-2) target proteins are detected using mass spectrometry methods. Exemplary Mass spectroscopy methods include, but are not limited to, MALDI-TOF-MS (U.S. Pat. Nos. 6,387,628 and 6,281,493, each of which is herein incorporated by reference); ESI oa TOF (LCT, Micromass) (See e.g., U.S. Pat. No. 6,002,127, herein incorporated by reference); ion trap mass spectrometry (U.S. Pat. Nos. 5,572,025, 5,696,376, 5,399,857, 5,420,425, each of which is herein incorporated by reference); ion trap/time-of-flight mass spectrometry; quadrupole and triple quadrupole mass spectrometry (U.S. Pat. No. 5,789,747, herein incorporated by reference); Fourier Transform (ICR) mass spectrometry (U.S. Pat. Nos. 3,937,955 and 4,755,670, each of which is herein incorporated by reference); and magnetic sector mass spectrometry.

In yet other embodiments, HERV-K(HML-2) target proteins are detecting using fluorescence in situ hybridization (FISH) in which antibody probes are contacted with whole cells or organisms.

In still further embodiments, cell free translation methods are utilized. For example, in some embodiments, cell-free translation methods from Ambergen, Inc. (Boston, Mass.) are utilized. Ambergen, Inc. has developed a method for the labeling, detection, quantitation, analysis and isolation of nascent proteins produced in a cell-free or cellular translation system without the use of radioactive amino acids or other radioactive labels. Markers are aminoacylated to tRNA molecules. Potential markers include native amino acids, non-native amino acids, amino acid analogs or derivatives, or chemical moieties. These markers are introduced into nascent proteins from the resulting misaminoacylated tRNAs during the translation process.

One application of Ambergen's protein labeling technology is the gel free truncation test (GFTT) assay (See e.g., U.S. Pat. No. 6,303,337, herein incorporated by reference). In some embodiments, this assay is used to screen for truncation mutations in proteins expressed from HERV-K(HML-2) targets. In the GFTT assay, a marker (e.g., a fluorophore) is introduced to the nascent protein during translation near the N-terminus of the protein. A second and different marker (e.g., a fluorophore with a different emission wavelength) is introduced to the nascent protein near the C-terminus of the protein. The protein is then separated from the translation system and the signal from the markers is measured. A comparison of the measurements from the N and C terminal signals provides information on the fraction of the molecules with C-terminal truncation (i.e., if the normalized signal from the C-terminal marker is 50% of the signal from the N-terminal marker, 50% of the molecules have a C-terminal truncation).

### D. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of cancer being present) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

### E. Kits

In yet other embodiments, the present invention provides kits for the detection and characterization of cancer. In some embodiments, the kits contain antibodies specific for a cancer marker (e.g., HERV-K(HML-2) targets), in addition to detection reagents and buffers. In other embodiments, the kits contain reagents specific for the detection of mRNA or cDNA (e.g., oligonucleotide probes or primers). In other embodiments, the kit contains reagents specific for detecting DNA. In preferred embodiments, the kits contain all of the components sufficient and/or necessary to perform a detection assay, including all controls, directions for performing assays, and any necessary software for analysis and presentation of results.

F. In vivo Imaging

In some embodiments, in vivo imaging techniques are used to visualize the presence of or expression of cancer markers in a subject (e.g., a human or non-human mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of the present invention are useful in the diagnosis of cancers that express the cancer markers of the present invention (e.g., cancer). In vivo imaging is used to visualize the presence of a marker indicative of the cancer. Such techniques allow for diagnosis without the use of an unpleasant biopsy. The in vivo imaging methods of the present invention are also useful for providing prognoses to cancer patients. For example, the presence of a marker indicative of cancers likely to metastasize can be detected. The in vivo imaging methods of the present invention can further be used to detect metastatic cancers in other parts of the body.

In some embodiments, reagents (e.g., antibodies) specific for the cancer markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin One 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific cancer marker of the present invention, to insure that the antigen binding site on the antibody will be protected. The antigen is separated after labeling.

In still further embodiments, in vivo biophotonic imaging (Xenogen, Almeda, C A) is utilized for in vivo imaging. This real-time in vivo imaging utilizes luciferase. The luciferase gene is incorporated into cells, microorganisms, and animals (e.g., as a HERV-K RNA protein with a cancer marker of the present invention). When active, it leads to a reaction that emits light. A CCD camera and software is used to capture the image and analyze it.

### G. Antibodies

The present invention provides isolated antibodies. In preferred embodiments, the present invention provides monoclonal antibodies that specifically bind to an isolated polypeptide comprised of at least five amino acid residues of the cancer markers described herein (e.g., HERV-K(HML-2) targets). These antibodies find use in the diagnostic methods described herein. For example, in some embodiments, where the HERV-K(HML-2) target protein expresses a portion of each HERV-K(HML-2) gene antibodies In other embodiments, wherein the expressed protein differs from wild-type protein (e.g., by truncation, structure, etc.), one or more antibodies are used to differentiate the modified form from the native form of the protein. For example, to detect truncations, two antibodies may be used, a first that binds to a shared region of the mutant and native form of the protein and a second that binds to the portion that is found only in the native form.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a cancer marker of the present invention (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### III. Drug Screening

In some embodiments, the present invention provides drug screening assays (e.g., to screen for anticancer drugs). The screening methods of the present invention utilize cancer markers identified using the methods of the present invention (e.g., including but not limited to, HERV-K(HML-2) targets). For example, in some embodiments, the present invention provides methods of screening for compounds that alter (e.g., decrease) the expression of cancer marker genes. The compounds or agents may interfere with transcription. The compounds or agents may interfere with mRNA produced from: HERV-K(HML-2) (e.g., by RNA interference, antisense technologies, etc.). The compounds or agents may interfere with pathways that are upstream or downstream of the biological activity of the HERV-K(HML-2) target. In some embodiments, candidate compounds are antisense or interfering RNA agents (e.g., oligonucleotides) directed against cancer markers. In other embodiments, candidate compounds are antibodies or small molecules that specifically bind to a cancer marker regulators or expression products of the present invention and inhibit its biological function.

In one screening method, candidate compounds are evaluated for their ability to alter cancer marker expression by contacting a compound with a cell expressing a cancer marker and then assaying for the effect of the candidate compounds on expression. In some embodiments, the effect of candidate compounds on expression of a cancer marker gene is assayed for by detecting the level of cancer marker mRNA expressed by the cell. mRNA expression can be detected by any suitable method. In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to cancer markers of the present invention, have an inhibitory (or stimulatory) effect on, for example, cancer marker expression or cancer marker activity, or have a stimulatory or inhibitory effect on, for example, the expression or activity of a cancer marker substrate. Compounds thus identified can be used to modulate the activity of target gene products (e.g., cancer marker genes) either directly or indirectly in a therapeutic protocol, to elaborate the biological function of the target gene product, or to identify compounds that disrupt normal target gene interactions. Compounds that inhibit the activity or expression of cancer markers are useful in the treatment of proliferative disorders, e.g., cancer, particularly lymphoma, leukemia and breast cancer.

In one embodiment, the invention provides assays for screening candidate or test compounds that are substrates of a cancer marker protein or polypeptide or a biologically active portion thereof. In another embodiment, the invention provides assays for screening candidate or test compounds that bind to or modulate the activity of a cancer marker protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In one embodiment, an assay is a cell-based assay in which a cell that expresses a cancer marker mRNA or protein, or biologically active portion thereof is contacted with a test compound, and the ability of the test compound to the modulate cancer marker's activity is determined. Determining the ability of the test compound to modulate cancer marker activity can be accomplished by monitoring, for example, changes in enzymatic activity, destruction or mRNA, or the like.

The ability of the test compound to modulate cancer marker binding to a compound, e.g., a cancer marker substrate or modulator, can also be evaluated. This can be accomplished, for example, by coupling the compound, e.g., the substrate, with a radioisotope or enzymatic label such that binding of the compound, e.g., the substrate, to a cancer marker can be determined by detecting the labeled compound, e.g., substrate, in a complex.

Alternatively, the cancer marker is coupled with a radioisotope or enzymatic label to monitor the ability of a test compound to modulate cancer marker binding to a cancer marker substrate in a complex. For example, compounds (e.g., substrates) can be labeled with ¹²⁵I, ³⁵S ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a cancer marker substrate) to interact with a cancer marker with or without the labeling of any of the interactants can be evaluated. For example, a microphysiorneter can be used to detect the interaction of a compound with a cancer marker without the labeling of either the compound or the cancer marker (McConnell et al. Science 257:1906-1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and cancer markers.

In yet another embodiment, a cell-free assay is provided in which a cancer marker protein or biologically active portion thereof is contacted with a test compound and the ability of the test compound to bind to the cancer marker protein, mRNA, or biologically active portion thereof is evaluated. Preferred biologically active portions of the cancer marker proteins or mRNA to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

In another embodiment, determining the ability of the cancer marker protein or mRNA to bind to a target molecule can be accomplished using real-time Biomolecular Interaction Analysis (BIA) (see, e.g., Sjolander and Urbaniczky, Anal. Chem. 63:2338-2345 [1991] and Szabo et al. Curr. Opin. Struct. Biol. 5:699-705 [1995]). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize cancer markers, an anti-cancer marker antibody or its target molecule to facilitate separation of complexed from non-complexed forms of one or both of the proteins, as well as to accommodate automation of the assay. Binding of a test compound to a cancer marker protein, or interaction of a cancer marker protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-cancer marker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or cancer marker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of cancer markers binding or activity determined using standard techniques. Other techniques for immobilizing either cancer markers protein or a target molecule on matrices include using conjugation of biotin and streptavidin. Biotinylated cancer marker protein or target molecules can be prepared from biotin-NHS (N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with cancer marker protein or target molecules but which do not interfere with binding of the cancer markers protein to its target molecule. Such antibodies can be derivatized to the wells of the plate, and unbound target or cancer markers protein trapped in the wells by antibody conjugation. Methods for detecting such complexes, in addition to those described above for the GST-immobilized complexes, include immunodetection of complexes using antibodies reactive with the cancer marker protein or target molecule, as well as enzyme-linked assays which rely on detecting an enzymatic activity associated with the cancer marker protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit. 11: 141-8 [1998]; Hageand Tweed J. Chromatogr. Biomed. Sci. App 1 699:499-525 [1997]). Further, fluorescence energy transfer may also be conveniently utilized, as described herein, to detect binding without further purification of the complex from solution.

The assay can include contacting the cancer markers protein, mRNA, or biologically active portion thereof with a known compound that binds the cancer marker to form an assay mixture, contacting the assay mixture with a test compound, and determining the ability of the test compound to interact with a cancer marker protein or mRNA, wherein determining the ability of the test compound to interact with a cancer marker protein or mRNA includes determining the ability of the test compound to preferentially bind to cancer markers or biologically active portion thereof, or to modulate the activity of a target molecule, as compared to the known compound.

To the extent that cancer markers can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, cancer markers protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent W0 94/10300; each of which is herein incorporated by reference), to identify other proteins, that bind to or interact with cancer markers (“cancer marker-binding proteins” or “cancer marker-bp”) and are involved in cancer marker activity. Such cancer marker-bps can be activators or inhibitors of signals by the cancer marker proteins or targets as, for example, downstream elements of a cancer markers-mediated signaling pathway.

Modulators of cancer markers expression can also be identified. For example, a cell or cell free mixture is contacted with a candidate compound and the expression of cancer marker mRNA or protein evaluated relative to the level of expression of cancer marker mRNA or protein in the absence of the candidate compound. When expression of cancer marker mRNA or protein is greater in the presence of the candidate compound than in its absence, the candidate compound is identified as a stimulator of cancer marker mRNA or protein expression. Alternatively, when expression of cancer marker mRNA or protein is less (i.e., statistically significantly less) in the presence of the candidate compound than in its absence, the candidate compound is identified as an inhibitor of cancer marker mRNA or protein expression. The level of cancer markers mRNA or protein expression can be determined by methods described herein for detecting cancer markers mRNA or protein.

A modulating agent can be identified using a cell-based or a cell free assay, and the ability of the agent to modulate the activity of a cancer markers protein can be confirmed in vivo, e.g., in an animal such as an animal model for a disease (e.g., an animal with lymphoma, leukemia or breast cancer, or metastatic lymphoma, leukemia or cancer; or an animal harboring a xenograft of a lymphoma, leukemia or breast cancer cancer from an animal (e.g., human) or cells from a cancer resulting from metastasis of a lymphoma, leukemia or breast cancer cancer (e.g., to a lymph node, blood, bone, bone marrow, or liver), or cells from a lymphoma, leukemia or breast cancer cancer cell line.

This invention further pertains to novel agents identified by the above-described screening assays (See e.g., below description of cancer therapies). Accordingly, it is within the scope of this invention to further use an agent identified as described herein (e.g., a cancer marker modulating agent, an antisense cancer marker nucleic acid molecule, a siRNA molecule, a cancer marker specific antibody, or a cancer marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### IV. Cancer Therapies

In some embodiments, the present invention provides therapies for cancer (e.g., lymphoma, leukemia or breast cancer). In some embodiments, therapies directly or indirectly target cancer markers (e.g., HERV-K(HML-2) target).

### A. Antisense and RNAi Therapies

In some embodiments, the present invention targets the expression of cancer markers. For example, in some embodiments, the present invention employs compositions comprising oligomeric antisense or RNAi compounds, particularly oligonucleotides (e.g., those identified in the drug screening methods described above), for use in modulating the function of nucleic acid molecules encoding cancer markers of the present invention, ultimately modulating the amount of cancer marker expressed.

### 1. RNA Interference (RNAi)

In some embodiments, RNAi is utilized to inhibit HERV-K(HML-2) target function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g., 21-23 nucleotides in length), and have a base-paired structure characterized by two nucleotide 3′-overhangs. Following the introduction of a small RNA, or RNAi, into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments. In some embodiments, RNAi oligonucleotides are designed to target the HERV-K(HML-2) proteins.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001;15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by referencce) describe the use of a type of DNA array called a scanning array to find accessible sites in mRNAs for designing effective siRNAs. These arrays comprise oligonucleotides ranging in size from monomers to a certain maximum, usually synthesized using a physical barrier (mask) by stepwise addition of each base in the sequence. Thus the arrays represent a full oligonucleotide complement of a region of the target gene. Hybridisation of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol. Biol. 2005 May 13; 348(4):883-93, J Mol. Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

### 2. Antisense

In other embodiments, HERV-K(HML-2) protein expression is modulated using antisense compounds that specifically hybridize with one or more nucleic acids encoding cancer markers of the present invention. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of cancer markers of the present invention. In the context of the present invention, “modulation” means either an increase (stimulation) or a decrease (inhibition) in the expression of a gene. For example, expression may be inhibited to potentially prevent tumor proliferation.

It is preferred to target specific nucleic acids for antisense. “Targeting” an antisense compound to a particular nucleic acid, in the context of the present invention, is a multi-step process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a cancer marker of the present invention. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A few genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in U.S. Patent WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂—[wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₁ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]2, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. ° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-5-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

### B. Genetic Therapies

The present invention contemplates the use of any genetic manipulation for use in modulating the expression of cancer markers of the present invention. Examples of genetic manipulation include, but are not limited to, gene knockout (e.g., removing a HERV-K(HML-2) gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter (e.g., an androgen-responsive promoter)).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Appl. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

### C. Antibody Therapy

In some embodiments, the present invention provides antibodies that target tumors that express a cancer marker of the present invention (e.g., HERV-K(HML-2) target protein). Any suitable antibody (e.g., monoclonal, polyclonal, or synthetic) may be utilized in the therapeutic methods disclosed herein. In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against a cancer marker of the present invention (e.g., HERV-K(HML-2)), wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technetium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeted a cancer marker of the present invention (e.g., HERV-K(HML-2)). Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

### D. Pharmaceutical Compositions

The present invention further provides pharmaceutical compositions (e.g., comprising pharmaceutical agents that modulate the expression or activity of HERV-K(HML-2) of the present invention). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more antisense compounds and (b) one or more other chemotherapeutic agents that function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### V. Transgenic Animals Expressing Cancer Marker Genes

The present invention contemplates the generation of transgenic animals comprising an exogenous cancer marker gene (e.g., HERV-K(HML-2)) of the present invention or mutants and variants thereof (e.g., truncations or single nucleotide polymorphisms). In preferred embodiments, the transgenic animal displays an altered phenotype (e.g., increased or decreased presence of markers) as compared to wild-type animals. Methods for analyzing the presence or absence of such phenotypes include but are not limited to, those disclosed herein. In some preferred embodiments, the transgenic animals further display an increased or decreased growth of tumors or evidence of cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad. Sci. USA 82:6927 [1985]). Transfection is easily and efficiently obtained by culturing the blastomeres on a monolayer of virus-producing cells (Stewart, et al, EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### EXPERIMENTAL EXAMPLES

The following examples are provided in order to demonstrate and further illustrate certain preferred embodiments and aspects of the present invention and are not to be construed as limiting the scope thereof.

### Example 1

### Detection of HERV-K(HML-2) Viral RNA in Plasma from HIV-Infected Patients

In work conducted in the course of development of the present invention HIV-1 and HCV-1 positive plasma samples were screened for the presence of HERV-K(HML-2) RNA in a RT-PCR using HERV-K pol specific primers. HERV-K(HML-2) viral RNA sequences were found in most HIV-1+ plasma samples (95.33%), but were rarely detected in HCV-1 patients (5.2%) or control subjects (7.69%). Other HERV-K(HML-2) viral segments of the RNA genome including gag, prt, and both env regions; surface (su) and transmembrane (tm) were amplified from HERV-K pol positive plasma of HIV-1 patients. Type-1 and type-2 HERV-K(HML-2) viral RNA genomes were found to coexist in same plasma of HIV-1 patients. These results suggest the HERV-K(HML-2) viral particles are induced in HIV-1 infected individuals.

A. HERV-K pol is Present in the Plasma of Patients With HIV-1

### Materials and Methods

Plasma-derived viral RNA samples were collected from patients infected with HIV-1, HIV-1/HCV-1, HCV-1 and seronegative control subjects, and screened for plasma-associated HERV-K RNA using HERV-K pol specific primers. The presence of HERV-K(HML-2) was confirmed using specific primers (Table 1.) (Medstrand P and Blomberg J: Characterization of novel reverse transcriptase encoding human endogenous retroviral sequences similar to type A and type B retroviruses: differential transcription in normal human tissues. J Virol 1993; 67:6778-6787; Andersson M, Lindeskog M, Medstrand P, Westley B, May F and Blomberg J: Diversity of human endogenous retrovirus class II-like sequences. J Gen Virol 1999; 80:255-260).

TABLE 1

Primers used for RT-PCR amplification of several HERV-K
regions and other controls

| | | | Tₘ | Size |
|---|---|---|---|---|
| Target region | Forward | Reverse | (° C.)^{≠} | (bp) |
| HERV-K gag | G1:5′-AGAAGGAAAAGGTCCAG | G2:5′-AGACTTGTATCTGGCCT | 55 | 437 |
| | AATTA-3′ | CAACT-3′ | | |
| HERV-K prt | P1:5′-GACTATAAAGGCGAAAT | P2:5′-AGGTGAGAACGAAGGCT | 58 | 805 |
| | TC-3′ | CAA-3′ | | |
| HERV-K pol | P3:5′-TCCCCTTGGAATACTCC | P4:5′-CATTCCTTGTGGTAAAA | 50 | 297 |
| | TGTTTTYGT-3′ | CTTTCCAYTG-3′ | | |
| HERV-K su | ES1:5′-AGAAAAGGGCCTCCAC | ES2:5′-ACTGCAATTAAAGTAA | 52 | 1100 |
| | GGAGATG-3′ | AAATGAA-3′ | | 1392 |
| HERV-K tm | ET1:5′-GCTGTAGCAGGAGTTG | ET2:5′-TAATCGATGTACTTCC | 50 | 462 |
| | CATTG-3′ | AATGGTC-3′ | | |
| HERV-K U5- | U5:5′-AAATCTCTCGTCCCACC | L2:5′-CATTCCTTGTGGTAAAA | 50 | 5135 |
| | TTAC-3′ | CTTTCCAYTG-3′ | | |
| pol | | | | |
| HERV-H pol | 5′-TTAGAACCTCTCATTTCCTT | 5′-CTTGATGTGTAGGGAAGGG | 57 | 126 |
| | TCCATC-3′ | AGG-3′ | | |
| β-actin RNA | 5′-GCGCGGCTACAGCTTCA-3′ | 5′-TCTCCTTAATGTCACGCACG | 58 | 58 |
| | | AT-3′ | | |

^{≠}Annealing temperature: The annealing step in the PCR reaction was performed 5 to 8 ° C. below the lowest Tm of the subset of primers for each reaction.

Reverse transcription PCR(RT-PCR) was performed using the One-Step RT-PCR kit (Qiagen, Valencia, Calif.) according to the manufacturer's instructions. Five μL of Viral RNA equivalent to 14 μL of plasma were reverse transcribed at 50° C. for 30 min. The PCR was performed in 40 cycles, each consisting of 94° C. for 1 min; an annealing step 5° C. to 8° C. below the Tₘ of the primers for 1 min and an extension step of 1 min per 0.5 Kb (See Table 1.).

### Results

HERV-K pol was positive by RT-PCR in 95.33% of HIV-1 cases, but was rarely detected in HCV-1+ and HIV-1/HCV-1 seronegative control plasma samples (Table 2).

TABLE 2
Detection of HERV-K RNA in Plasma from Patients.

| Source of plasma tested | No. Positive | No. Tested | %. positive |
|---|---|---|---|
| HIV-1 positive patients | 184 | 193 | 95.33 |
| HIV-1/HCV positive patients | 15 | 15 | 100.00 |
| HCV positive patients | 1 | 19 | 5.20 |
| Seronegative blood donors | 1 | 13 | 7.69 |

HERV-K viral pol RNA amplified by RT-PCR using 5 μL of RNA extractions equivalent to 14 μL of plasma.
Positive results consist of at least 2 of 3 positive PCR replicates.

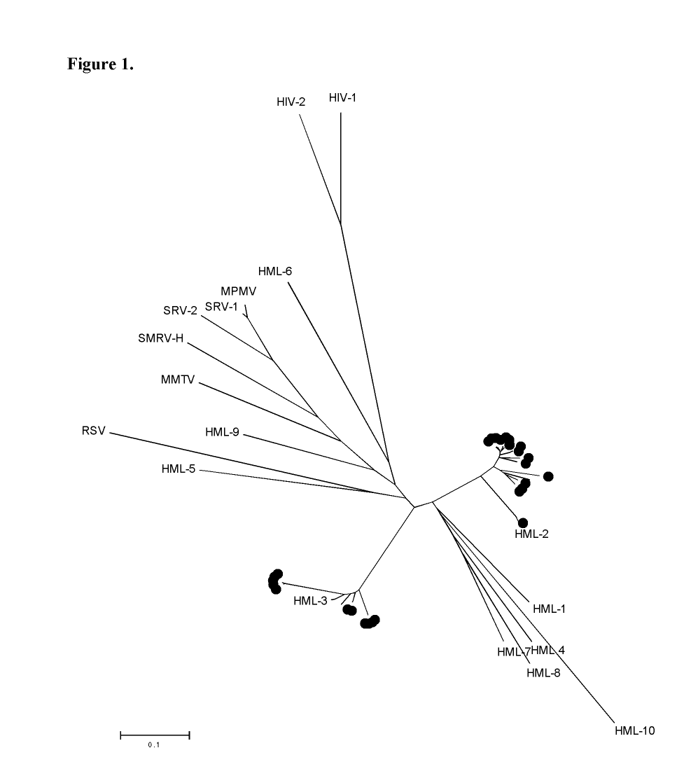

The authenticity of the PCR products was confirmed by sequencing. Neighbor-joining phylogenetic analysis of 30 HERV-K pol clonal sequences amplified from six different plasma samples confirmed the existence of the subfamily HERV-K(HML-2) (FIG. 1) The subfamily HERV-K(HML-3) was also co-amplified in all HIV-1 positive plasma samples studied. All the pol sequences amplified corresponding to HERV-K(HML-2) have intact open reading frames (ORFs).

B. HERV-K(HML-2) Transcripts Other Than pol are Present in the Plasma of Patients With HIV-1

### Materials and Methods

To rule out the possibility that only short pol RNA transcripts were present in plasma, different gene segments of the HERV-K(HML-2) viral RNA genome were amplified using the set of primers described in Table 1. Six plasma samples taken from HIV-1+, HIV-1+/HCV-1+, HCV-1+, and seronegative patients were used.

### Results

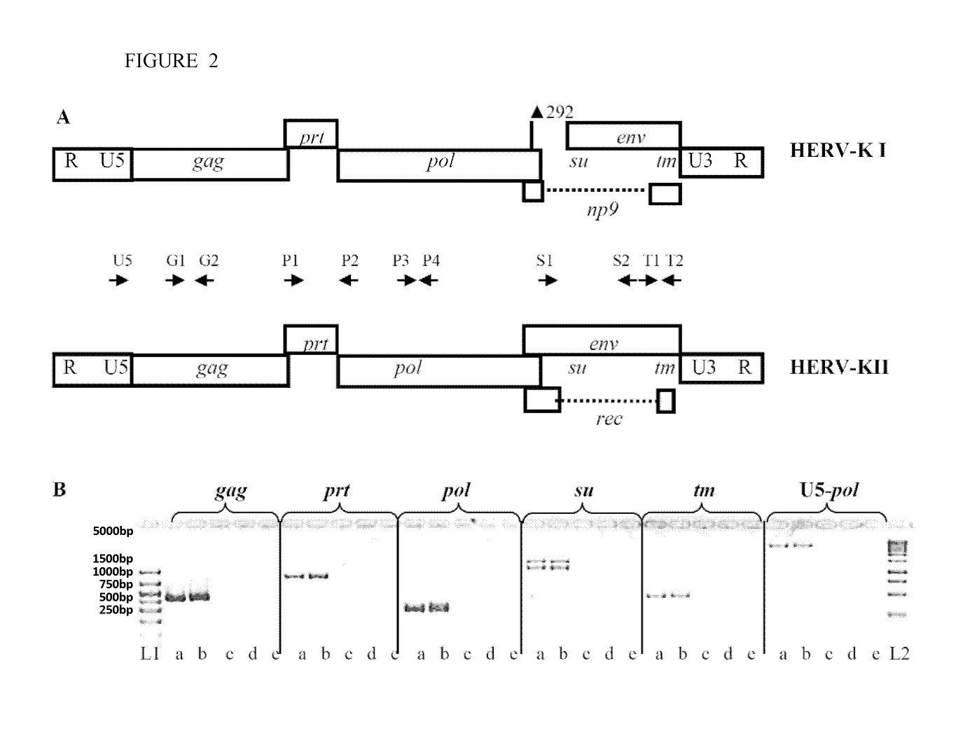

All HERV-K genes were amplified from HIV-1 seropositive patients but not from HCV-1+ patients or control subjects. (FIG. 2)

C. HERV-K mRNA Detected in the Plasma of Patients with HIV-1 are not Contaminants

### Materials and Methods

An amplification reaction without the reverse transcription step was also performed to eliminate the possibility of DNA contaminants in plasma samples.

### Results

β-actin primers that span spliced mRNA regions do not amplify in six HIV-1 RNA extractions, indicating that the HERV-K amplified is not a product of cellular RNA contamination. In addition, primers specific for HERV-H pol sequences, (Forsman A, Yun Z, Hu L, Uzhameckis D, Jern P, Blomberg J. Development of broadly targeted human endogenous gamma retroviral pol-based real time PCRs Quantitation of RNA expression in human tissues. J Virol Methods 2005; 129:16-30) previously found in plasma from rheumatoid arthritis patients, (Christensen T, Pederson L, Sorensen P D, Moller-Larsen A. A transmissible human endogenous retrovirus. AIDS Res Hum Retroviruses 2002; 18:861-866), did not amplify in HIV-1 RNA extracts.

D. Both Type-1 and Type-2 HERV-K(HML-2) Genomes are Present in Plasma from HIV-1 Patients

### Materials and Methods

The authenticity of the RT-PCR products was confirmed by sequencing. The size of the amplification product obtained with the env (su) primers was used to determine the type of HERV-K(HML-2) present in the amplification reactions.

To confirm the authenticity of the Real-Time RT-PCR reactions and determine the HERV-K subtypes activated in these HIV-1+ plasma samples, amplicons were cloned in the TA cloning vector pCR4 (Invitrogen, Carlsbad, Calif.) and sequenced. The cDNA sequences were assembled and aligned using the BioEdit platform.

### Results

A 292 bp deletion in type-1 viruses gives raise to a 1105 bp amplification product. On the other hand, HERV-K type-2 genomes are characterized by a 1397 bp amplicon. The amplification of env (su) showed both type-1 and type-2 HERV-K(HML-2) genomes to be present in plasma samples from HIV-1 patients (FIG. 1.). (Ono M, Yasunaga T, Miyata T and Ushikubo H: Nucleotide sequence of human endogenous retrovirus genome related to the mouse mammary tumor virus genome. J Virol 1986; 60:589-598; Lower R, Tonjes R, Korbmacher C, Kurth R and Lower J: Identification of a Rev-related protein by analysis of spliced transcripts of the human endogenous retroviruses HTDV/HERV-K. J Virol 1995; 69:141-149).

E. Full-length HERV-K RNA Genomes are Present in HIV-1+ Patients

### Materials and Methods

A longer region of the HERV-K viral genome was amplified.

### Results

By using a forward primer that spans the U5 RNA segment and a reverse primer that anneals to pol, a 5135 full length HERV-K genome was detected in 4 of 6 HIV-1 positive plasma samples (FIG. 1.). These results indicate that full-length HERV-K RNA genomes are present in HIV-1+ individuals. To protect the RNA genomes from abundant serum RNAses, retroviruses have preserved the gag gene to encode the matrix, capsid, and nucleocapsid structures, which is a pre-requisite for particle formation. (Blank A, Dekker C, Schieven G, Sugiyama R and Thelen M: Human body fluid ribonucleases: detection, interrelationships and significance. Nucleic Acids Symp Ser 1981; 10:203-209). Presence of HERV-K viral particles in the circulating blood of HIV-1 infected individuals provides the rationale for detection in plasma of antibodies reactive to HERV-K. (Lower R, Lower J and Kurth R: The viruses in all of us: characteristics and biological significance of human endogenous retrovirus sequences. Proc Natl Acad Sci USA 1996; 93:5177-5184; Vogetseder W, Dumfahrt A, Mayersbach P, Schonitzer D and Dierich M: Antibodies in human sera recognizing a recombinant outer membrane protein encoded by the envelope gene of the human endogenous retrovirus K. AIDS Res Hum Retroviruses 1993; 9:687-694).

The only HERV-K subfamily known to produce viral particles is HERV-K(HML-2). ((Bannert N and Kurth R: Retroelements and the human genome: new perspectives on an old relation. Proc Natl Acad Sci USA 2002;101 Suppl 2:14572-14579; Simpson G, Patience C, Lower R, Tonjes R, Moore H, Weiss R and Boyd M: Endogenous D-type (HERV-K) related sequences are packaged into retroviral particles in the placenta and possess open reading frames for reverse transcriptase. Virology 1996; 222:451-456; Bieda K, Hoffmann A and Boller K: Phenotypic heterogeneity of human endogenous retrovirus particles produced by teratocarcinoma cell lines. J Gen Virol 2001; 3:591-596; Boller K, Konig H, Sauter M, Mueller-Lantzsch N, Lower R, Lower J and Kurth R: Evidence that HERV-K is the endogenous retrovirus sequence that codes for the human teratocarcinoma-derived retrovirus HTDV. Virology 1993; 1:349-353). In the course of development of the present invention HERV-K(HML-2) RNA genomes have been observed in HIV-1-infected plasma samples. Sequencing analyses of the proviruses that are expressed in HIV-1 positive patients indicates the activation of 32 of 128 HERV-K(HML-2) members with sequence similarities between 98.5% and 100%. These proviruses have flanking LTRs, and are not HERV-K(HML-2) fragments. Compared to the 18 type-2 elements expressed in HIV-1 patients, many sequences were similar to K108, K109, K115 and K113 viruses. (Barbulescu M, Turner G, Seaman M, Deinard A, Kidd K and Lenz J: Many human endogenous retrovirus K (HERV-K) proviruses are unique to humans. Curr Biol 1999; 9:861-868; Turner G, Barbulescu M, Su M, Jensen-Seaman M, Kidd K and Lenz J: Insertional polymorphisms of full-length endogenous retroviruses in humans. Curr Biol 2001; 11:1531-1535). However, some sequences are more than 2% divergent from these proviruses. Recent evidence suggests that humans retain a pool of replication-competent viruses. (Belshaw R, Dawson A L, Woolven-Allen J, Redding J, Burt A, Tristem M. Genomewide screening reveals high levels of insertional polymorphism in the human endogenous retrovirus family HERV-K(HML2): implications for present-day activity. J Virol 2005; 79:12507-12514).

### Example 2

### Quantification of HERV-K(HML-2) RNA in Plasma from Patients with HIV-Associated Lymphomas, and Non-HIV-Associated Lymphomas, Leukemia and Breast Cancer

Reverse transcriptase genes are among the most conserved regions of many retroviruses, including HERVs (McClure M A, Johnson M S, Feng D F, Doolittle R F. Sequence comparisons of retroviral proteins: relative rates of change and general phylogeny, Proc. Natl. Acad. Sci. U.S.A. 85 (1988), pp. 2469-2473). The HERV-K family is subdivided into 10 groups (HML-1 to HML-10) (Nelson P, Carnegie P, Martin J, Davari E., Hooley P, Roden D, Rowland-Jones S, Warren P, Astley J, Murray P. Demystified human endogenous retroviruses, Mol. Pathol. 56 (2003), pp. 11-18). The HERV-K(HML-2) subfamily is the phylogenetically most recent form of the HERVs. It is transcriptionally active, and is responsible for the production of HERV-K viral particles. In turn, the gag gene is the most well conserved of all HERV-K(HML-2) members.

### Materials and Methods

RNA Extractions from Plasma Samples

Plasma collected in EDTA was stored at −70° C. in 1 mL aliquots for up to 12 years after collection. A subset of earlier samples was collected as part of a study of nucleic acid sequence based assay (NASBA) used to determine the viral burden in HIV patients. Viral RNA was extracted from frozen plasma samples using the QIAamp viral RNA mini kit following the manufacturer's procedure (Qiagen, Valencia, Calif.). All samples were treated with 200 units of DNAse (Roche, Indianapolis, Ind.) for 2 hours prior to RNA extraction to eliminate contamination from cellular DNA. RNA extracted from 140 μL of plasma was eluted in 50 μL RNAse-free water.

### Primer Selection

Primers were designed to amplify a 214 bp HERV-K(HML-2) gag product. This set of primers is KgagF 5′-AGC AGG TCA GGT GCC TGTA ACA TT-3′, and KgagR 5′-TGG TGC CGT AGG ATT AAG TCT CCT-3′.

### Construction of HER V-K RNA Standards

HERV-K(HML-2) gag cDNA was amplified from the plasma of a single HIV-1 infected individual using the primers described. The amplicon was cloned in plasmid pCR2.1 (Invitrogen, Carlsbad, Calif.). After confirming the authenticity of the plasmid by sequencing, the construct was linearized with SacI, that cuts a sequence downstream from the PCR insert and the T7 priming site. HERV-K RNA standards were produced using T7 RNA polymerase and the competitor construction kit (Ambion, Austin, Tex.). In vitro RNA standards were treated with RNAse-free DNAse for 2 hours at 37° C. and purified twice by ETOH precipitation in the presence of 3M sodium acetate, pH 5.2 at −20° C. The purified in vitro RNA was quantified spectrophotometrically at 260 nm and diluted serially to obtain RNA concentrations ranging from ˜3×10⁰ to ˜3×10⁹ copies/mL

### Quantitation of HER V-K(HML-2) RNA Copy Number/mL by Sybr Green Real-Time RT-PCR

To measure HERV-K(HML-2) RNA Copy Number/μL, Real-Time (RT)-PCR was performed using the QuantiTect Sybr Green RT-PCR kit (Qiagen, Valencia, Calif.). Five μL of extracted RNA, or of standard RNA, and 0.2 μM each of sense and antisense primer were used in a final 20 μL master mix volume. A reverse transcription step of 20 min at 50° C. was included prior to PCR. PCR reactions consisted of 50 cycles with conditions as follows: 94° C. for 15 sec; 50° C. for 20 sec; 72° C. for 30 sec; and a collection data step, 85° C. for 5 sec. Fluorescence captured at 85° C. was determined to be absent of signal generated by primer dimmers or other non-specific product. All samples were run in triplicate, and the RNA standards were run in duplicate.

Data were collected and recorded by the iCycler iQ software (Bio-Rad, Milpitas, Calif.) and expressed as a function of the threshold cycle (C_{T}), which represents the number of cycles at which the fluorescent intensity of the Sybr Green dye is significantly above the background fluorescence. C_{T} is directly correlated to the log₁₀ copy number/mL of the RNA standards. RNA copies were extrapolated from standard curves (C_{T} vs. log₁₀ copy number/mL) representing at least seven-point serial dilutions of standard RNA (10¹ to 10⁹ copies/mL). RNA standards were used as calibrators to the relative quantification of product generated in the exponential phase of the amplification curve for Real-Time RT-PCR. The results were accepted for standard curves with correlation coefficients greater than 0.95.

Representative plasma RNA extractions were performed by standard PCR to assure the absence of contaminating DNA. Positive HERV K amplicons were confirmed by melting curve analyses and ethidum bromide staining in agarose gels to visualize the 214 bp product.

### Results

A. Detection of HERV-K(HML-2) RNA in Plasma from Patients with HIV-associated Lymphomas, Non-HIV-associated Lymphomas, Leukemia and Breast Cancer

Viral HERV-K RNA was detected by Real Time RT-PCR in plasma samples from HIV-1 patients that developed large cell lymphoma (LCL), central nervous system (CNS) lymphoma, other forms of lymphoma and/or Hodgkin's disease (HD). Viral titers were also measured in HIV-1 negative patients with chronic lymphatic leukemia (CLL), acute myeloid leukemia (AML), and breast cancer (BC). Plasma from patients with HIV who did not develop lymphoma, and HIV negative controls, was also investigated. The HERV-K RNA viral burden in these conditions is shown in Table 3.

TABLE 3
Detection of HERV-K(HML-2) RNA
in Patient and Control Plasma

| Source of plasma tested | No. Positive | No. Tested | %. positive |
|---|---|---|---|
| Healthy individuals | 7 | 28 | 25 |
| HIV-1 | 24 | 34 | 67 |
| HIV/AIDS positive Large | 29 | 30 | 96 |
| cell lymphoma | | | |
| HIV negative Large cell | 19 | 19 | 100 |
| lymphoma | | | |
| HIV/AIDS CNS lymphoma | 5 | 5 | 100 |
| HIV Hodgkin's Disease | 5 | 5 | 100 |
| HIV negative Hodgkin's | 2 | 2 | 100% |
| Disease | | | |
| HIV+ T cell leukemia | 1 | 1 | 100% |
| Acute myeloid leukemia* | 0 | 11 | 0 |
| Chronic lymphatic leukemia | 5 | 5 | 100 |
| Breast Cancer | 43 | 47 | 91 |

HERV-K viral gag RNA was amplified by RT-PCR using 5 μL of RNA extractions. Positive results consisted of at least 2 of 3 positive PCR replicates
*Plasma collected with heparin

B. Quantification of HERV-K(HML-2) RNA Titers in Patient and Control Plasma

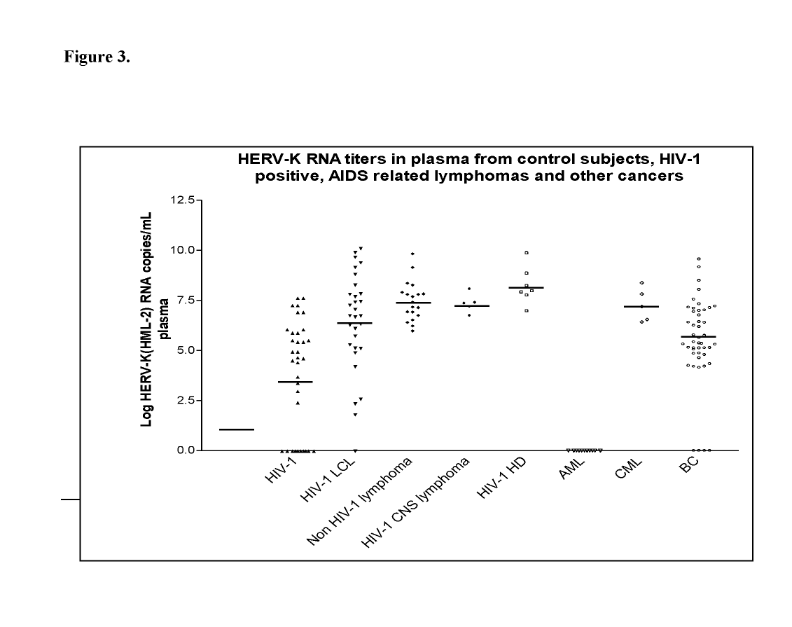

To further explore HERV K RNA detection in plasma from patients described in Table 1, the levels of the respective viral burdens were measured in the cited clinical conditions. The HERV-K RNA titers are shown in FIG. 3. The Log₁₀ HERV-K RNA titers in patients with lymphoma (HIV+, HIV−, healthy controls and HIV patients without lymphoma are shown in FIG. 3. (ANOVA p<0.0001) Statistical difference between the HERV-K RNA titers in different groups were tested using the one-way ANOVA test in the SPSS Platform. A significant p-value resulting from a one-way ANOVA test indicates that the HERV-K titers from one group are differentially increased in at least one of the groups analyzed. If more than two groups were analyzed, post hoc tests were applied to determine which specific pair/pairs are differentially increased.

Patients with lymphoma have increased viral RNA titers (Log₁₀HERV-K RNA/mL median=7.38) compared to healthy individuals (Log₁₀ HERV-K RNA/mL median=0.78, p<0.0001), HIV positive individuals with no lymphoma (Log₁₀ HERV-K RNA/mL median=3.82, p<0.0001), and breast cancer (BC) patients (Log₁₀ HERV-K RNA/mL median=5.50, p<0.0001). No significant difference was observed in the HERV-K RNA viral burden in patients with different types of lymphoma (p=0.346) including Hodgkin's lymphoma, large cell lymphoma, Burkitt's lymphoma, T-cell lymphoma, small cell indolent lymphoma, CNS lymphoma, and chronic lymphocytic leukemia. Interestingly, HERV-K RNA was found in high titers in chronic lymphocytic leukemia (CLL) patients but was undetectable in acute myeloid leukemia (AM) patients. Thus, the present data shows that there are high viral loads (as high as 10⁹) to HML-2 in the plasma of patients with HIV-associated lymphomas. In addition non-HIV patients with lymphoma and other cancers also have high viral burdens of these viruses.

C. Quantification of HERV-K(HML-2) RNA Titers in HIV+Hodgkin's Disease and Non-Hodgkin's Lymphoma in Patients with Remission after Chemotherapy

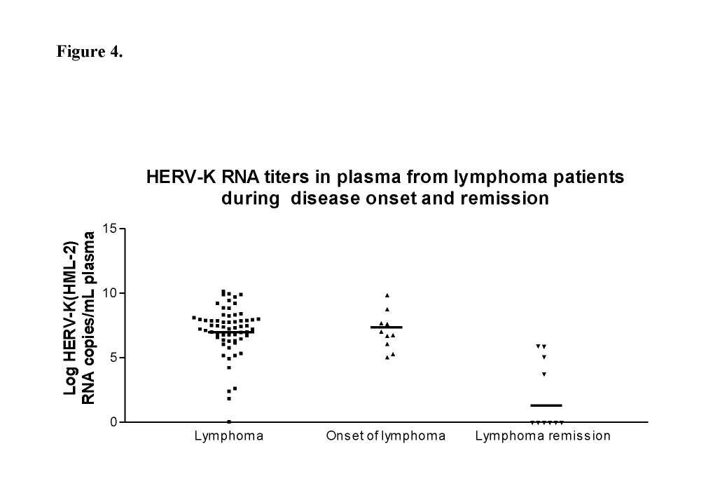

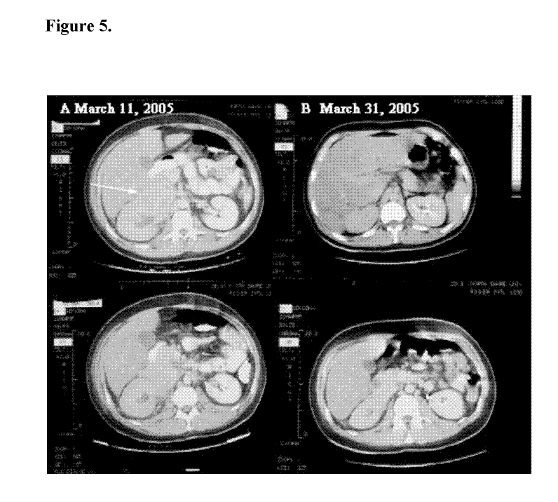

HERV-K(HML-2) RNA titers were quantified in plasma samples of 10 individuals who responded to chemotherapy with tumor regression and/or complete remission with chemotherapy and/or radiation treatment. HERV-K titers were quantified during a period of 2 to 7 years before development of neoplastic disease in HIV patients and then after treatment. Clinical information was obtained from chart review. The Log₁₀ HERV-K RNA/mL titers observed in these patients were measured without knowledge of the treatment course or activity of HIV disease. The HERV-K(HML-2) titers found immediately before, and at the peak of the appearance of lymphoma (Log₁₀ HERV-K RNA median=7.13), were significantly higher than the titers observed after complete or partial remission (FIG. 4). (Log₁₀ HERV-K(HML-2) RNA/mL median=2.70, p<0.001)

D. HERV-K(HML-2) RNA Titers Correlate Treatment with Foscarnet (PFA) Treatment

HIV-1/AIDS patients may be co-infected with cytomegalovirus (CMV) and develop viral retinitis. (Masur H, Whitcup S M, Cartwright C, Polis M, Nussenblatt R. Advances in the management of AIDS-related cytomegalovirus retinitis. Ann Intern Med. 1996 Jul. 15; 125(2):126-36.) Three patients with AIDS-related lymphoma were followed for a period of 3 months to 5 years before and after CMV retinitis and Foscarnet (PFA) treatment. Because PFA reduces tumor size in certain AIDS-related lymphoproliferative disorders (Schmidt W, Anagnostopoulos I, Scherubl H. Virostatic therapy for advanced lymphoproliferation associated with the Epstein-Barr virus in an HIV-infected patient. N Engl J. Med. 2000 Feb. 10; 342(6):440-1; Schneider U, Ruhnke M, Delecluse H J, Stein H, Huhn D. Regression of Epstein-Barr virus-associated lymphoproliferative disorders in patients with acquired immunodeficiency syndrome during therapy with foscarnet. Ann Hematol. 2000 April; 79(4):214-6.), the effect of PFA on the HERV-K(HML-2) viral load was determined.

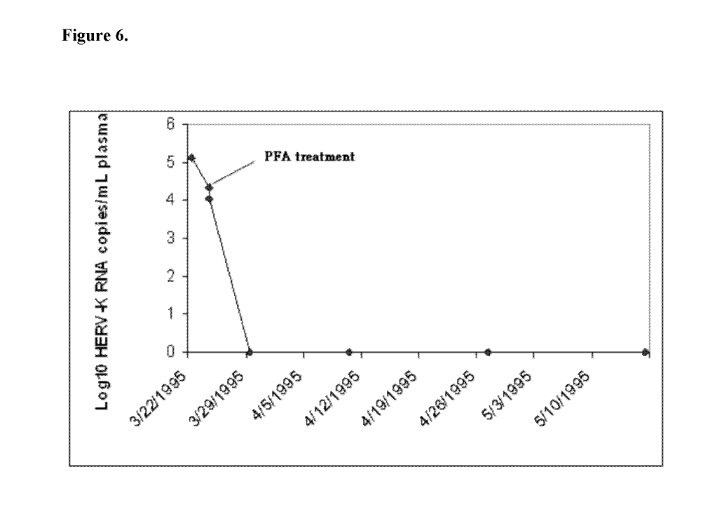

One patient experienced sudden onset of fever, chills and abdominal pain, and a large mass in the right kidney as shown in the CAT scan below (FIG. 5A, upper). The patient underwent biopsy of the kidney mass that revealed large cell lymphoma (LCL). The patient also had severe CMV retinitis with CMV viremia. The patient was started on PFA for CMV retinitis on day 2, and was not felt to be a candidate for treatment of the lymphoma because of the CMV infection. As the patient improved on PFA he was to start chemotherapy. The patient's abdominal pain improved however and the mass was no longer palpable. A repeat CAT scan showed reduction in the tumor mass (FIG. 5B). FIG. 5 shows a mass in the right kidney in the two left panels. The mass regressed 20 days after the start of PFA as shown in the two right panels. The patient later went on to have further complications of CMV, but remained on PFA. The patient ultimately died 4 months after the last abdominal CAT. The autopsy revealed a small nodule in the right kidney with LCL cells. In this patient the HERV-K(HML-2) RNA burden was suppressed 5 days after PFA treatment was begun (FIG. 6). HERV-K(HLM-2) RNA titers remained undetectable thereafter. Thus, in this patient undetectable HERV-K levels correlated with PFA treatment (p<0.001)

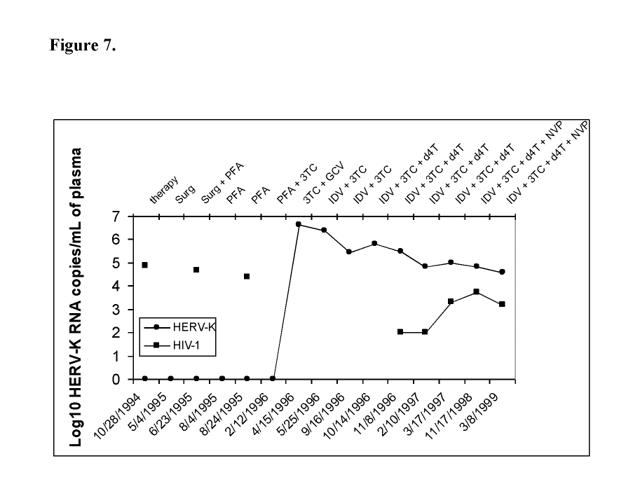

Two additional patients underwent PFA treatment after diagnosis of CMV retinitis. One had LCL, and the other had central nervous system (CNS) lymphoma. In both patients PFA was used for several weeks but discontinued after CMV treatment failure. PFA was changed to Gancyclovir (GCV). The HERV-K(HML-2) RNA titers were quantified before, during and after PFA treatment. The first patient (FIG. 7) complained of severe abdominal pain for several months and was noted to have enlarged lymph nodes in the abdomen. These were biopsied and showed LCL. The patient refused therapy for the lymphoma, and had the onset of worsening abdominal pain. Blood cultures revealed CMV. A CAT scan of the abdomen showed enlarged mesenteric nodes. The patient was started on PFA. The abdominal pain began to improve and the patient was later taken to surgery to confirm the presence of the lymphoma. At surgery the lymphoma was not observed. The patient remained on PFA over the next 18 months. When HAART became available the patient was switched to antivirals as shown in FIG. 7. The patient was now intolerant of PFA and it was discontinued. Shortly after stopping PFA the HERV-K(HML-2 RNA viral titers rose dramatically and persisted. The patient's HIV remained in control over the following years. The patient died suddenly, but had continued abdominal node enlargement on a CAT scan prior to death. FIG. 7 shows the elevation in HERV-K viral load after PFA was stopped in this patient who experienced spontaneous regression of lymphoma on PFA earlier.

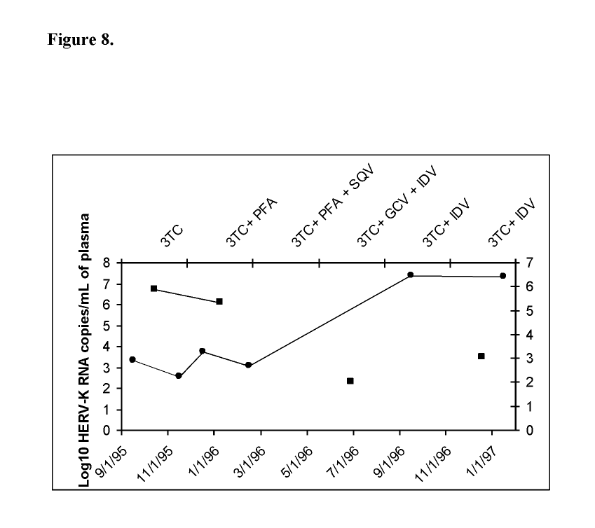

In a second patient with CNV viremia and CNS lymphoma that had improved after radiation treatment a similar elevation in HERV-K viral load was observed avter PFA was stopped due to intolerance of the medication (FIG. 8).

Because significant levels of the HERVs HML-2 were observed in HIV patients, this assay was used to quantify the viral load of these agents in HIV lymphoma plasma using a quantitative viral load assay based upon the env gene rather than the pol gene of HML-2. Using this assay, elevated levels of HML-2 were observed in patients with HIV associated lymphoma. These levels were as high as 10⁹ in some patients. One patient who had the highest viral load discovered at the height of Burkitt's lymphoma severity had also had large cell lymphoma 5 years earlier. After going into remission from Burkitt's lymphoma, he then developed myelodysplastic syndrome and then acute leukemia. As the leukemia developed, his HML-2 viral load rose significantly. In another patient, who presented with HIV and CMV retinitis and a mass in the kidney that was lymphoma, spontaneous remission of lymphoma with the use of foscarnet to treat CMV retinitis was noted. This was associated with a significant clearance of HERV-K HML-2 from the plasma of this patient. Hence, there is a significant decrement in HRV-K HML-2 viral load in patients who are successfully treated with chemotherapy for HIV associated lymphoma.

### Example 3

### Identification of Recombinant HERV-K (HML-2) env Sequences as a Marker for Viral Replication

### Materials and Methods

### RT-PCR, Cloning and Sequencing

Supernatants from the breast cancer cell line K151 was fractioned by sucrose sedimentation, and the viral RNA was extracted from the particulate using the QIAamp viral RNA mini kit following the manufacturer's instructions (Qiagen, Valencia, Calif.). RNA was also extracted from HIV1/AIDS patients and 5 HIV negative women with breast cancer who had significant HERV K viral loads in plasma.

RNA extracted from 140 μL of plasma was eluted in 50 μL RNAse-free water. The full-length env surface (SU) gene was amplified using the One-Step RT-PCR kit (Qiagen, Valencia, Calif.) with the primers

| | |
|---|---|
| ES1: | 5′AGAAAAGGGCCTCCACGGAGATG-3′ |
| and | |
| ES2: | 5′ACTGCAATTAAAGTAAAAATGAA-3′ |

that generates a ˜1351 bp amplification product in HERV-K(HML-2) type-2 elements. A 292 bp deletion in HERV-K(HML-2) type-1 led to the amplification of a RT-PCR product ˜1059 bp. A portion of the env transmembrane (TM) sequence was amplified with the primers

| | |
|---|---|
| ET1: | 5′GCTGTAGCAGGAGTTGCATTG-3′ |
| and | |
| ET2: | 5′TAATCGATGTACTTCCAATGGTC-3′ |

that generates a ˜464 bp product. The amplification products were cloned in the TA cloning vector, pCR2.1 (Invitrogen, Carlsbad, Calif.) and sequenced. The sequences were assembled using the BioEdit platform. The nucleotide and deduced amino acid sequences were aligned using the Clustal W multiple alignment program.

### Identification of HERV-K(HML-2) Proviruses Using Blast to Search the HERVd and NCBI Databases

Because of divergence in the HERV-K(HML-2) env gene (which differs between 1% to 20% among all proviruses in this subfamily) sequences in the HERVd database (Paces J, Pavlicek A, Zika R, Kapitonov V V, Jurka J, Paces V. HERVd: the Human Endogenous RetroViruses Database: update. Nucleic Acids Res. 2004 Jan. 1;32 (Database issue:D50) were BLAST (Basic Local Alignment Search Tool) searched to determine which (HERV-K(HML-2) are detected in plasma samples. The analyses included three elements (AF006332, K103 and K113) found exclusively in the NCBI database. The criterion of element identification was >99% sequence similarity. Open reading frames (ORFs) were calculated using translated-BLAST in the NCBI database. Alignments of cDNA and known proviruses were exported to the MEGA matrix (Kumar S, Tamura K, Nei M. MEGA3: Integrated software for Molecular Evolutionary Genetics Analysis and sequence alignment. Briefings in Bioinformatics 5:150-163, 2004). Phylogenetic trees were constructed by neighbor-joining, maximum parsimony, and maximum likelihood methods, using the statistical bootstrap test (1000 replicates) of inferred phylogeny and the kimura-2 parameter model. (ibid.) Using distance from the MEGA matrix, inter-subtype distances between HERV-K proviruses were calculated. The identification of HERV-K(HML-2) elements was confirmed by the clustering of the same provirus in a phylogenetic branch. HERV-K(HML-2) proviral sequences activated in HIV-1 infection were manually inspected for the presence of conserved elements in the long terminal repeats (LTRs) and reading frames and conserved motifs for all viral genes as described by Turner et al., 2001. (Turner G, Barbulescu M, Su M, Jensen-Seaman M I, Kidd K K, Lenz J. Insertional polymorphisms of full-length endogenous retroviruses in humans. Curr Biol. 2001 Oct. 2; 11 (19): 1531-5.).

### Tests for Recombination

Sequences were evaluated for potential recombinant events using several methods. First, the neighbor-joining tree for each data set was inspected. Recombination of large portions of different elements may generate branches with unresolved topology, resulting in taxonomic units that either protrud far beyond the other taxa, or fell far short in comparison. Recombinant sequences were found in 25% of all the sequences amplified in HIV-1 patients, and in 50% in the breast cancer cell line K151. On these occasions recombinants were <99% similar to the closest element. Identification of the potential parent sequences and recombination sites were elucidated using RIP 2.0 (“Scanning the Database for Recombinant HIV-1 Genomes”, Siepel A C, Korbe B T, MS K710, Los Alamos National Laboratory, Los Alamos NM 87545. Part III of The Human Retroviruses and AIDS 1995 Compendium). The program uses a sliding window (200 bp in this study) that moves over an alignment containing the query sequence, and all the background sequences or identified elements. After the window has traversed from left to right it generates a recombination plot that describes the background representative that most nearly resembles the query sequence at all possible windows. Best matches are highlighted if they are significant by using an internal statistical test. Sequence similarity between the putative parent and query sequence at each side of the recombination site was visually verified.

### Results

Based on a 292 bp DNA fragment present in type-2 but not type-1 HERV-K(HML2) elements (12), a total of 400 type-1 and 200 type-2 clone sequences were obtained. Diversity in the nucleotide composition of the HERV-K(HML-2) family, and the presence of deletions or insertion mutations particular to each element, made phylogenetic reconstruction using the env gene suitable for the identification of the proviruses activated in the lymphoma and breast cancer patients. The best sequence similarity to HERV-K(HML-2) elements and their chromosomal location were determined for each clone together with the integrity of their reading frames. (See Tables 5. and 6. in Example 4. below). Despite a 292 bp deletion between pol and env which might be deleterious for the processing of the Pol-Env polyprotein, type-1 elements were observed to have an intact env ORFs for expression of the NP9 protein. HERV-K type-2 env sequences were detected in the plasma of lymphoma and breast cancer patients but rarely detected in Hodgkin's disease individuals.

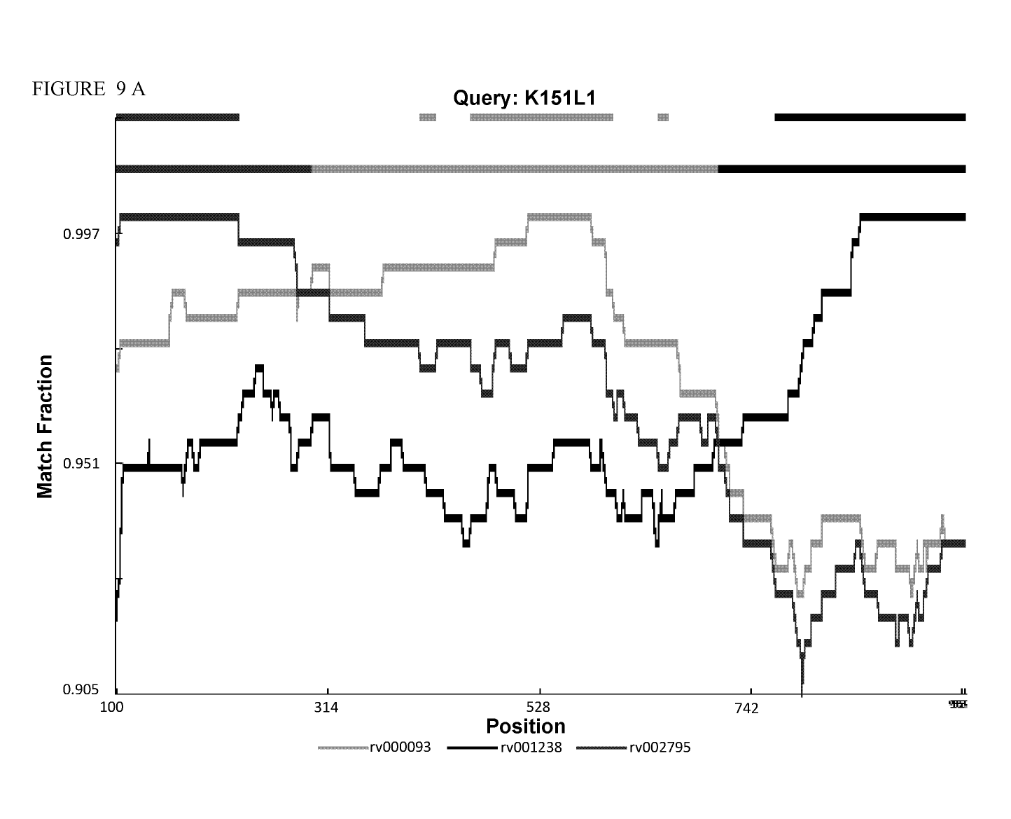

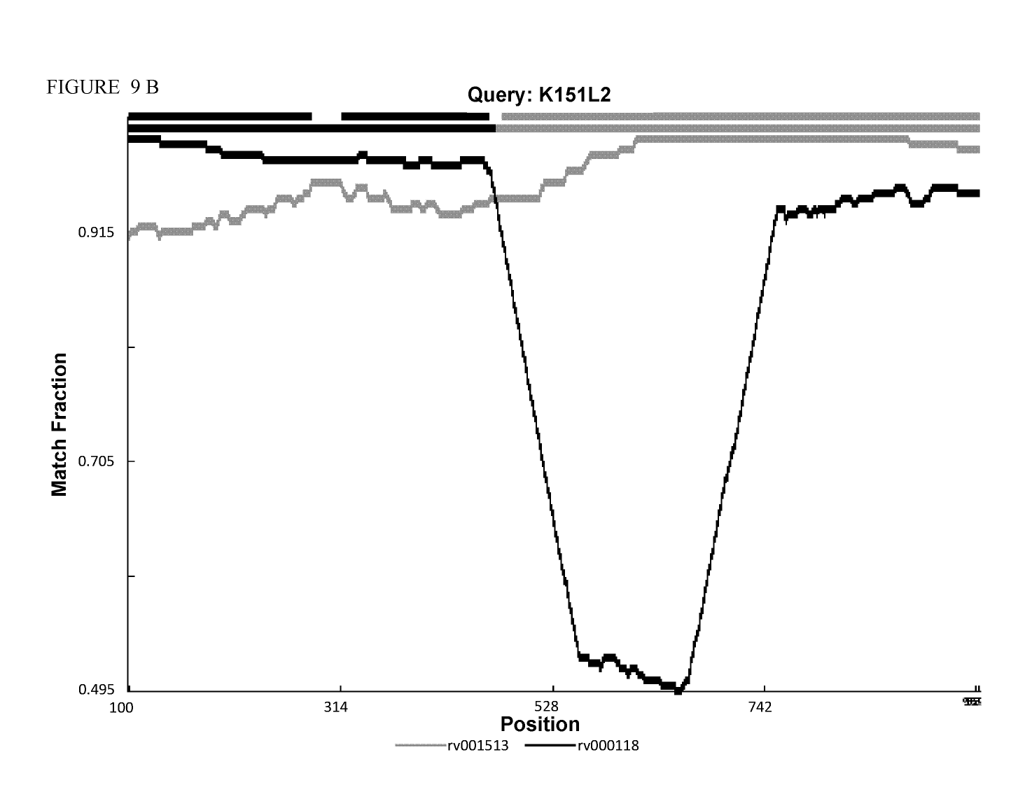

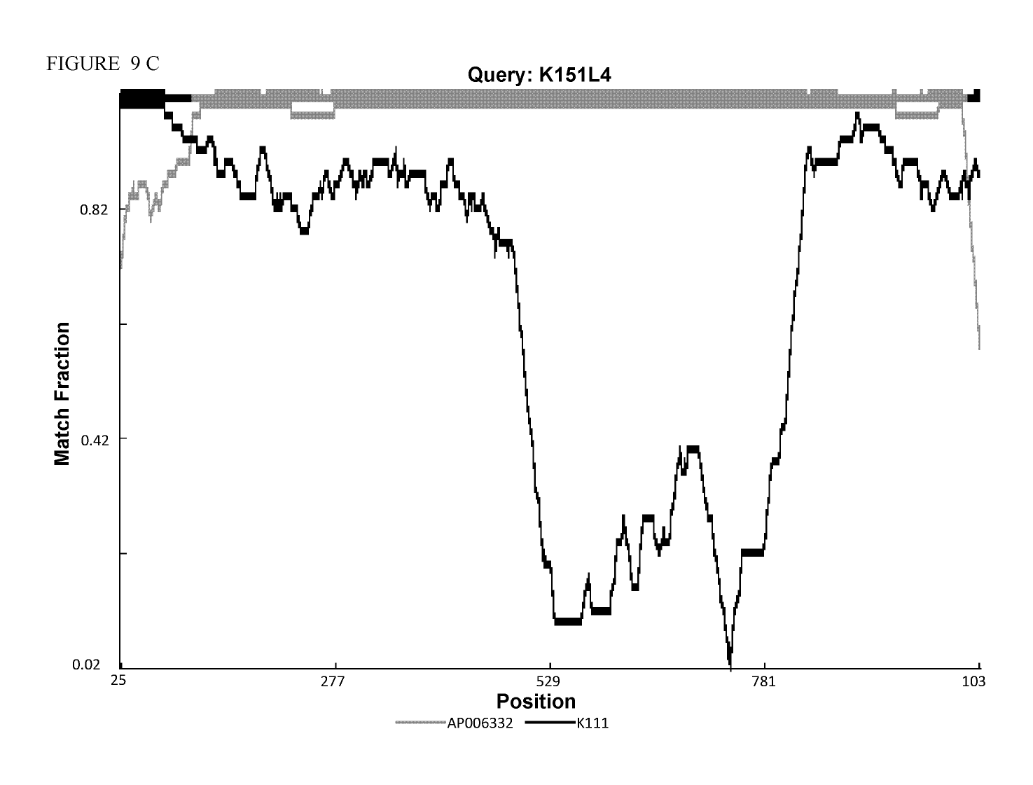

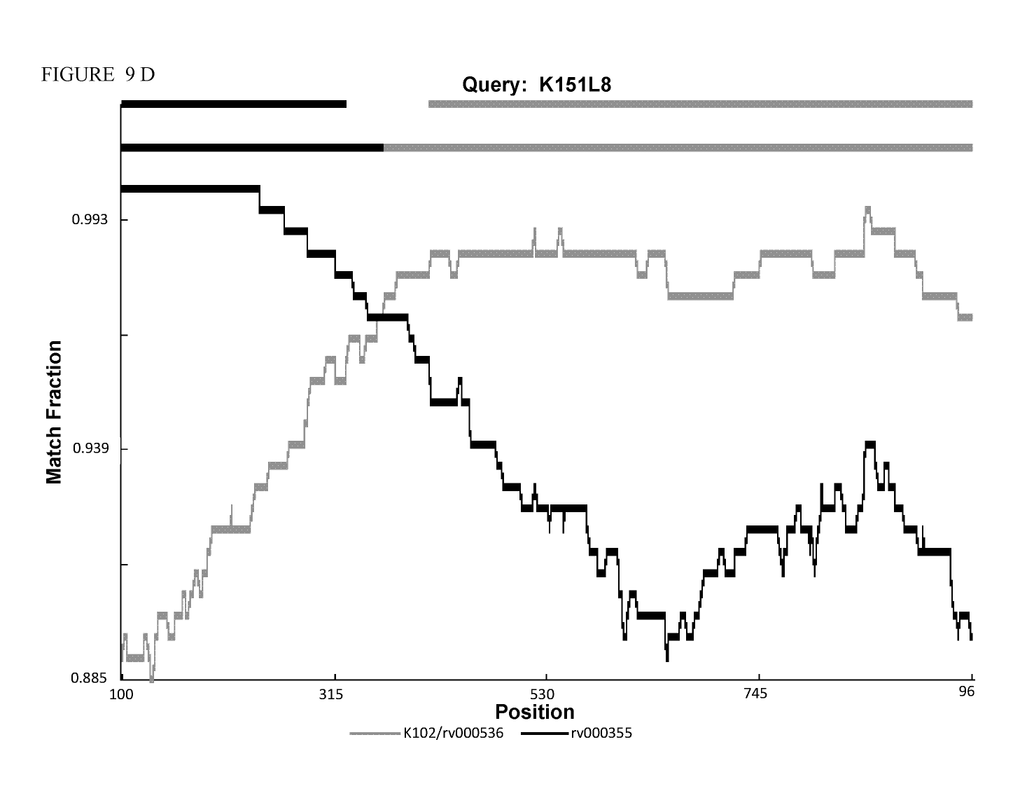

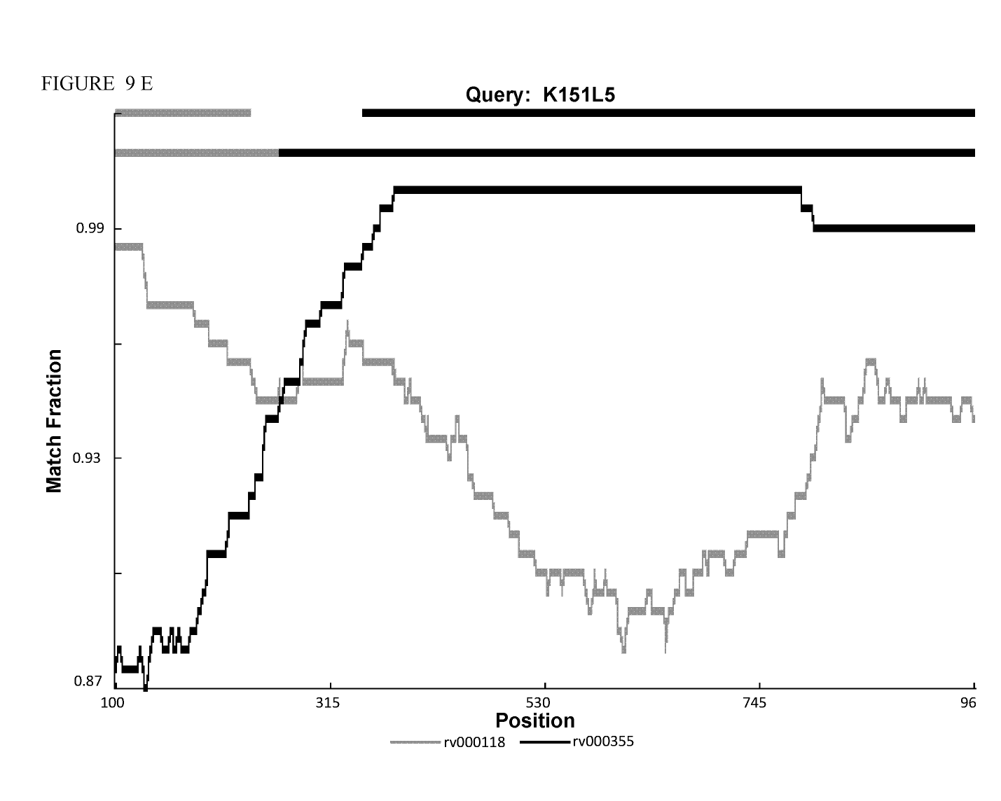

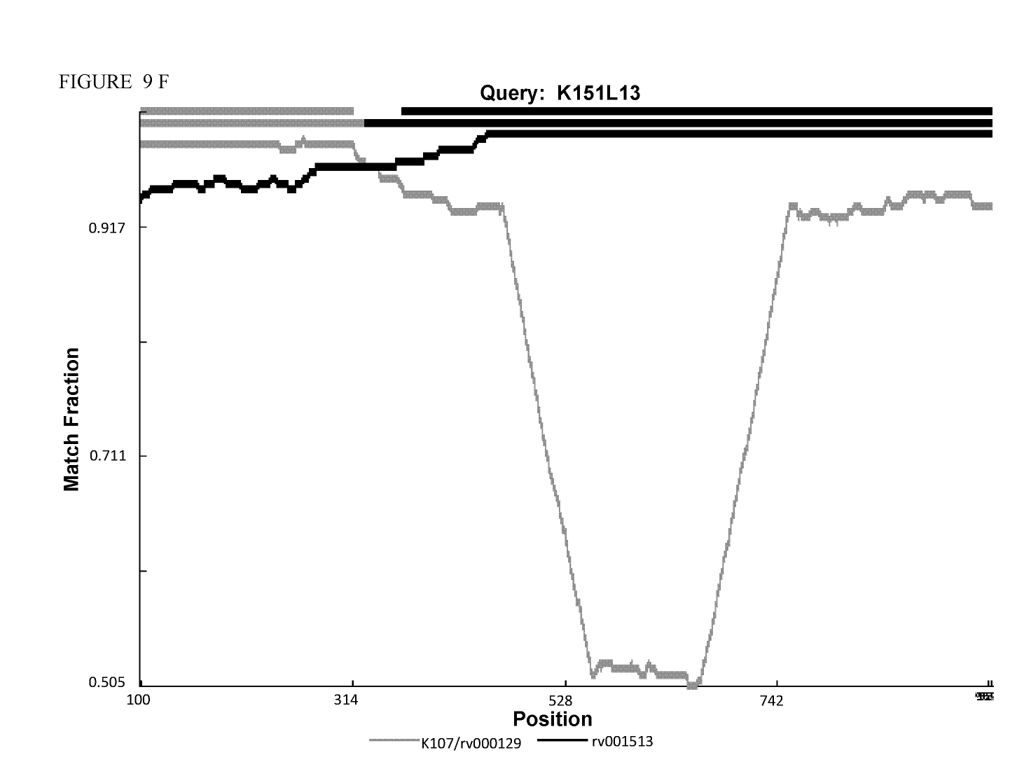

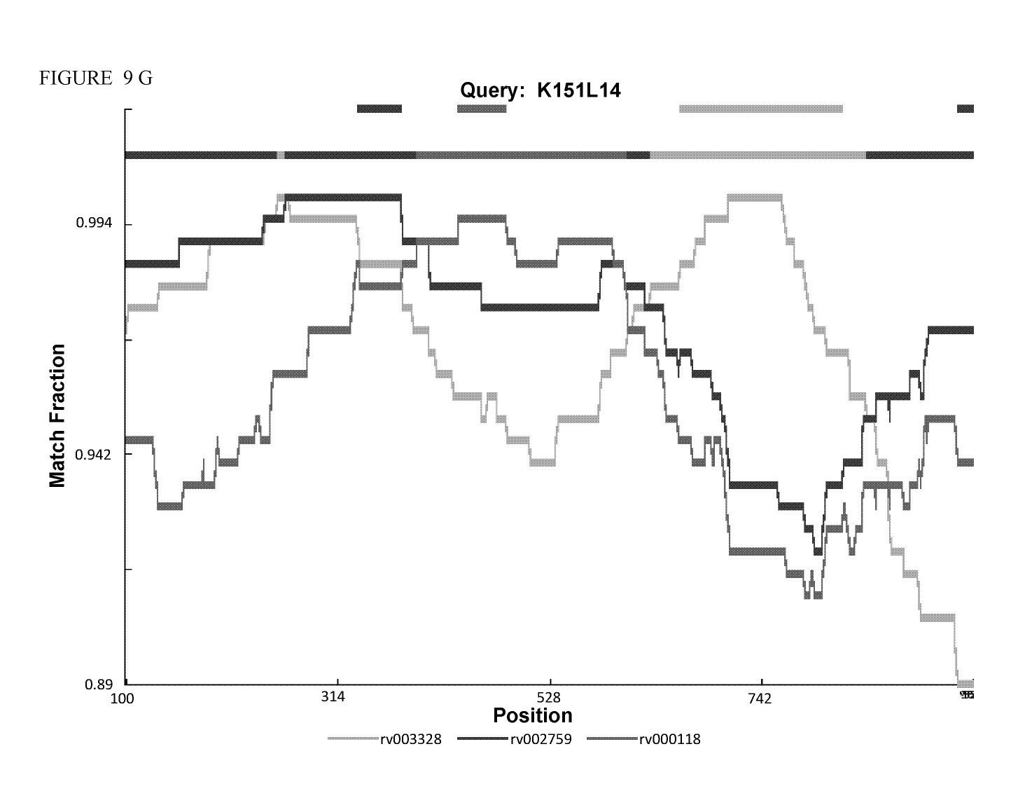

HERV-K RNA was isolated from supernatants of the breast cancer cell line K151. Detection of recombinant sequences was confirmed by RIP 2.0 recombination analyses (ibid.) that display statistical significant recombinant similarities between the ancestor sequences and the recombinant. Exemplary recombination plots of HERV-K(HML-2) env sequences from the K151 breast cancer cell line are shown in FIG. 9. The similarity between the query sequence and each background representative is plotted for each position of a ˜1000 bp sliding window. The Y axis represents the match fraction of each query sequence to each parental sequence (black and grey lines, respectively). A match fraction of 1 means 100% identity between the two. The representation of the recombinant clone query sequence is illustrated in the upper X axis (upper color line) Thick lines in the recombinant query sequence indicate significance in the best match at a 90% threshold level. Significant putative type-1/type-1 and multiple recombinant sequences are illustrated. The identification of the clone sequences of the putative recombinant clones is described. Recombinant sequences from the K151 cell line indicate replication of the HERV-K(HML-2) family. The sequences were reconstructed in a phylogenetic model aligned to distinct sequences isolated from plasma samples of breast cancer patients. The neighbor joining method, with bootstrapping of 1000 replicated different alignments used by the program, produces a phylogenetic tree showing evidence of recombinant sequences (those branches that do not cluster to identified viruses and protruded far beyond or fell short, compared to the other taxa).

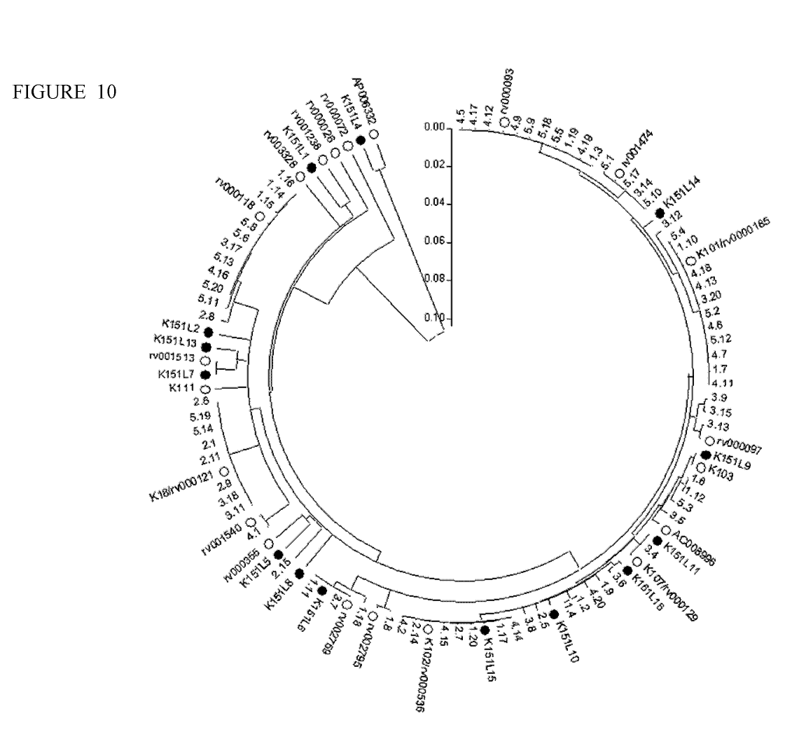

FIG. 10. shows a phylogenetic neighbor-joining tree of type-1 HERV-K(HML-2) env (SU) sequences amplified from breast cancer patients, and from the cell line K151. The tree is unrooted, with taxa arranged for a balanced shape. FIG. 10. depicts recombinant sequences K151L4, K151L1, K151L2, K151L3, K151L5, K151L8 and K151L14 configured in a phylogenetic tree. Branch distances were calculated using the Kimura 2-parameter model for uniformed distributed rates among nucleotide sites and 1000 bootstrap replicates. White circles represent reported HERV-K proviruses in the HERVd and NCBI database. Black circles represent K151 exogenous HERV-K env sequences, including the recombinants forms (K151L4, K151L1, K151L2, K151L3, K151L5, K151L8 and K151L14).

These results demonstrate that HERV-K recombination in the envelope gene produce new recombinant sequences that are of use in the determination of viral replication of HERV-K. Recombination occurs after a viral particle infects a cell and liberates two RNA strands, with each one reverse transcribed to cDNA by viral reverse transcriptase. Low affinity in reverse transcriptase recognition allows the enzyme to shift from one RNA strand to the other RNA, thereby creating a recombinant cDNA sequence that is then integrated to form a proviral form. Recombinant and non-recombinant sequences are then activated to produce RNA that is packaged into the viral particle and released from the cell. The percentage of amplified recombinant sequences correlates with the rate of viral replication; failure to find recombinant sequences may indicate slow or no replication. An increase in the degeneracy of the sequences (less than 99.5% similarity to any of the two ancestors) may be added evidence of replication rate. After one cycle of viral infection and replication, few or any mutations are introduced and the viral RNA is zero, one or two bases less identical to the progenitor. An increase in the number of mutations indicates that the viruses have replicated over a longer time interval, and passed through many cycles of infection, thereby creating RNA sequences much less similar to the original progenitor.

### Example 4

### HERV-K(HML-2) env Sequences in Blood Correspond to Different HERV-K(HML-2) Virions in Different Patients

The types of HML2 (type 1 and 2 viruses) that are present in the blood of patients with neoplastic disease were determined by amplification of viral envelope genes from patient sample, that were then sequenced to determine the different types of HML2 virions present.

### Methods and Materials

### Patients

Plasma from patients with very high HERV K viral loads who had HIV lymphoma (3 with HIV associated large cell lymphoma, 1 with HIV Burkitt's cell lymphoma, with HIV associated HD, and 1 with HIV associated T cell lymphoma), and HIV negative breast cancer (4 patients), HIV negative CLL (1 patient), and 2 HIV-negative Hodgkin's disease were selected for RNA extraction.

### RNA Extraction, PCR, Detection, Cloning and Sequencing

RNA was extracted from 140 ul of plasma that had been pretreated with 20 ul of Roche DNAse RNAse free (Roche, Manheim Germany 10776785001) for 2 hours. RNA was extracted using the Qiagen (Valencia, Calif.) QIAmp Viral RNA Mini Kit Cat #52906. 4-5 ul of RNA was then subjected to RT PCR using the either the Super Script One Step RT-PCR for long templates (Cat. No 11922-010 Carlsbad Calif) or the Qiagen OneStep RT PCR kit Cat 210210) using the following env primers:

KenvSUF AGAAAAGGGCCTCCACGGAGATG forward

KenvSUR TTCATTTTTACTTTAATTGCAGT reverse.

The following PCR protocol was utilized to amplify these products.
Initial RT step 42° C. 30 min
Then 9502 min
Then 40 cycles at:
   95° 30 sec
   42° 60 sec
68° 120 sec
final 73° extension 15 min

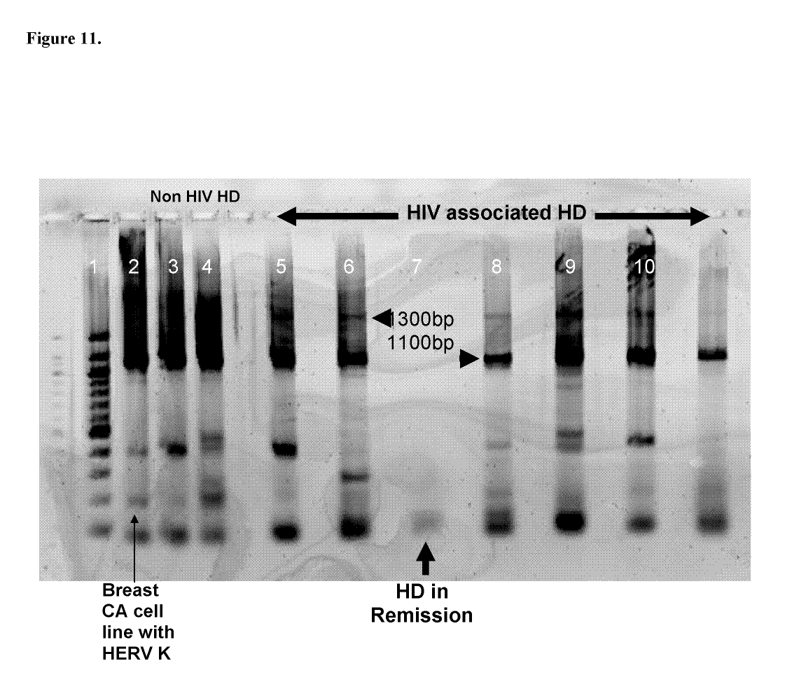

This program and its primers amplify the 1105 bp and/or approximately 1300 bp HERV-K(HML-2) env DNA. Products of amplification were resolved on a 1.5% agarose gel and bands appearing at mw 1100 and 1300 were cut from the gel. FIG. 11 shows a gel from plasma templates from patients with Hodgkin's disease. The bands cut from the gel were subjected to a high speed spin and amplified DNA (4-5 ul) was cloned using the TOPO TA Cloning Kit for sequencing PCR using the TOPO vector (cat. No. 45-0030 Invitrogen, Carsbad, C A). DNA was extracted from bacteria grown on LB broth using the Eppendorf Fast Plasmid Mini kit 0032007.653. Extacted DNA was sequenced in the University of Michigan DNA sequencing core (Ann Arbor, Mich.) and subjected to analysis in the BLAST program of the NCBI and in the HERVd data base.

### Results

FIG. 11 shows a 1.5% agarose gel depicting the RT-PCR products amplified from plasma RNA taken from different patients with HIV associated HD (lanes 4-11), and non HIV HD lanes 2 and 3 using env specific sequences. Lane 1 (control) shows amplified RNA by RT-PCR from the supernatant of a breast cancer cell line K151 that produces HML2 viral particles. The 1105 bp product is from HML2 type 1 virus envelope and the 1300 bp product, which is less distinct, is from the approximately 1350 bp product of HML2 type 2 virions. In one patient in remission from HD (lane 7), who had a non detectable HERV K viral load using gag primers, the viral envelope products could not be demonstrated by RT PCR using the env primers.

Multiple HERV-K(HML-2) viral envelope sequences were identified in each patient sample. All patients with high viral loads demonstrated with gag primers had significant env bands using the env primers in RT PCR. Table 4 shows env sequences that were observed by analyzing the RT-PCR products that were amplified and cloned from the env region of individual patients. The sequence of these clones was matched to HERV sequences deposited in the HERVd database which is an on going new data base expressly for the deposition of sequences related to HERVs. This database uses distinct numbers to designate unique HERVs. Up to 20 clones were sequenced in each patient using the methods described above. The different viral types are shown in the Table 4.

TABLE 4
HERV-K(HML-2) Viral Types

Patient

| plasma | Number | Viral types designated by the HERVd data base | | | | | | | | | | | |
|---|---|---|---|---|---|---|---|---|---|---|---|---|---|
| sample | clones | 185 | 182 | 129 | 97 | 355 | 536 | 93 | 118 | 121 | 1474 | 2759 | other |
| HD12 | 18 | 7 | | 4 | 1 | 1 | 1 | 1 | 1 | 2 | | | |
| HD13 | 20 | 2 | 5 | 1 | | 2 | 1 | | | 1 | 4 | 3 | |
| HD 14 | 17 | 8 | | 1 | | 2 | 4 | | 2 | | | | |
| HD15 | 14 | 1 | | 5 | | | 4 | 1 | | | 3 | | |
| HD16 | 19 | 2 | | 3 | 1 | | | | 2 | 1 | 1 | 3 | 4 |
| LCL6 | 15 | 1 | | 3 | 3 | | 2 | | | 1 | 1 | | 3 |
| LCL10 | 13 | 1 | 1 | 3 | 2 | | | 2 | 4 | | | | |

While the observed env sequences were homologous to the viruses in the HERV d database, there is significant divergence in the sequences from the known numbered genomic HERVs (down to 95% homology). This is illustrated in the sequences from one patient with lymphoma as shown in Table 5, and a second patient with Hodgkin's Disease (Table 6.).

TABLE 5
Extent of Homology Between Observed and Reported HERV-K(HML-2)
env Sequences in a Patient With Lymphoma

| | HERVd viral | Identity to known |
|---|---|---|
| Sequence | number | sequence |
| 727571LCL6.02 | >>rv 001540 | 95.294% identity |
| 727575LCL6.06 | >>rv 001540 | 95.265% identity |
| 727570LCL6.01 | >>rv 001540 | 95.656% identity |
| 727578LCL6.09 | >>rv 000129 | 98.486% identity |
| 727574LCL6.05 | >>rv 000129 | 98.549% identity |
| 727577LCL6.08 | >>rv 000129 | 97.378% identity |
| 727579LCL6.10 | >>rv 001474 | 99.005% identity |
| 727582LCL6.13 | >>rv 000536 | 98.192% identity |
| 727576LCL6.07 | >>rv 000536 | 99.365% identity |
| 727573LCL6.04 | >>rv 000097 | 99.638% identity |
| 727580LCL6.11 | >>rv 000097 | 97.742% identity |
| 727572LCL6.03 | >>rv 000097 | 98.281% identity |
| 727587LCL6.18 | >>rv 000185 | 98.552% identity |
| 727588.LCL6.19 | >>rv 000185 | 98.552% identity |
| 727589.LCL6.20 | >>rv 000121 | 97.448% identity |

TABLE 6
Extent of Homology Between Observed and Reported HERV-K(HML-2)
env Sequences in a Patient With Hodgkin's Disease

| | HERVd viral | Identity to known |
|---|---|---|
| Sequence | number | sequence |
| 727703.-HD14.15 | >>rv 000118 | 99.277% identity |
| 727705.-HD14.17 | >>rv 000118 | |
| 727694.-HD14.06 | >>rv 000355 | 97.466% identity |
| 727708.-HD14.20 | >>rv 000355 | 98.915% identity |
| 727689.HD14.01 | >>rv 000536 | 97.658% identity |
| 7277063-HD-14.18 | >>rv 000536 | 94.846% identity |
| 727695.-HD14.07 | >>rv 000536 | 98.644% identity |
| 727696.-HD14.08 | >>rv 000536 | 95.204% identity |
| 727697.-HD14.09 | >>rv 000185 | 99.548% identity |
| 727698.-HD14.10 | >>rv 000185 | 99.278% identity |
| 727699.-HD14.11 | >>rv 000185 | 98.825% identity |
| 727700.HD-HD14.12 | >>rv 000185 | 98.917% identity |
| 727701.-HD14.13 | >>rv 000185 | 99.458% identity |
| 727702.-HD14.14 | >>rv 000185 | 99.097% identity |
| 727691.-HD14.03 | >>rv 000185 | 98.828% identity |
| 727707.HD14.19 | >>rv 000185 | 98.735% identity |
| 727704.HD-14.16 | >>rv 000129 | 98.374% identity |

Most HML-2 virions detected in HD were type 1, but both type 1 and 2 HML-2 sequences were found in patients with large cell lymphoma. In patients with multiple clones from the same virus, clonal variation was detected. This divergence is indicative of viral variation due to active replication of these viruses in such patients. In some of the env sequences amplified, greater divergence from the known sequences represented in the HERVd data base was observed. These variations proved to be recombinant sequences that code for active viral proteins, and are indicative of active replication of HML2 species that created these recombination events.

In non HIV breast cancer patients, similar viruses were observed with distinct patterns of distribution. The env products are highly represented in these cancer patients.

Thus, the env primers of the present invention can be used to amplify and quantify HERV-K(HML-2) viruses, for example, type 1, type 2 and recombinant variations, as well. The env region provides improved sequence substrates to subtype viruses in plasma because there is great diversity in the env region, and many of the differences in HERV-K(HML-2) virions occurs in env regions.

A higher degree of viral variation is indicative of active HML-2 viral subtype viral replication in these patients, which allows detection of the HERV-K(HML-2) subtype that is activated in each cancer, and serves as a marker of the presence of a particular cancer, or as a measure of the virulence and pathogenicity of an HERV-K)HML-2) associated cancer, or as an indicator of a response to therapy of such a cancer. In particular, screening for HERV-K(HML-2) subtypes will prevent iatrogenic virally-induced cancers in transfused patients and organ recipients.

### Example 5

### NASBA Assay for Quantification of Human Endogenous Retroviruses Type-K (HERV-K (HML-2)) Subtype 1 and 2 in Plasma Samples from Cancer Patients

The primers in Tables 7, 8 and 9 assay are used to detect and characterize HERV-K(HML-2) viral RNA in plasma samples from patients with HIV and HIV associated lymphomas, and non-HIV lymphomas and breast cancer, using nucleic acid sequence based amplification (NASBA).

### Materials and Methods

Three HERV-K(HML-2) regions are targeted for NASBA amplification. The gag region is conserved for all HERV-K(HML-2) subfamily, thus quantification of gag provides general HERV-K(HML-2) titers. Specific primers are designed to quantify type 2 viruses targeting the env region, deleted in type 1 viruses. A total of 6 primers are designed for each target. To amplify type 1 and not type 2 viruses a region in the env sequence that is consensual for type 1 viruses (95 to 100%), but nearly degenerate for type 2 viruses (only 85% similar), is selected.

TABLE 7

Sequence of primers and probes of the
HERV-K(HML-2) gag region

| | |
|---|---|
| Name | Sequence 5′-3′ |

| | |
|---|---|
| KgagRTF | AGCAGGTCAGGTGCCT |
| | GTAACATT |
| | (SEQ. ID. NO: 1) |
| KgagRTR | TGGTGCCGTAGGATTA |
| | AGTCTCCT |
| | (SEQ. ID. NO: 2) |
| Kgag probe 1 | AAGACCCAACCACCAG |
| | TAGCCTATCA |
| | (SEQ. ID. NO: 3) |

TABLE 8

Sequence of primers and probes for
HERV-K(HML-2) type −1 env viruses

| | |
|---|---|
| Name | Sequence 5′-3′ |

| | |
|---|---|
| Ktype1F | AGAAAAGGGCCTCCAC |
| | GGAGATG |
| | (SEQ. ID. NO: 4) |
| Ktype1R | CTCTCCCTAGGCAAAT |
| | AGGA |
| | (SEQ. ID. NO: 5) |
| Ktype1 probe 1 | ACGGAGATGGTAACAC |
| | CAGTCACATGGA |
| | (SEQ. ID. NO: 6) |

TABLE 9

Sequence of primers and probes for
HERV-K(HML-2) type -2 env viruses

| | |
|---|---|
| Name | Sequence 5′-3′ |

| | |
|---|---|
| Ktype2F | AGACACCGCAATCGAG |
| | CACCGTTGA |
| | (SEQ. ID. NO: 7) |
| Ktype2R | ATCAAGGCTGCAAGCA |
| | GCATACTC |
| | (SEQ. ID. NO: 8) |
| Ktype2 probe 1 | AAGTTGCCATCCACCA |
| | AGAAGGCAGA |
| | (SEQ. ID. NO: 9) |

### Construction of In Vitro RNA Transcripts

HERV-K(HML-2) gag and type-1 and type-2 env sequences are amplified from plasma of cancer patients by RT-PCR and cloned into vector PCR-4 TOPO (Invitrogen, Carlsbad, Calif.). Type 2 sequences contain the same pol-env region as type 1 transcripts plus the 292 bp env insertion (481 bp) lacking in type 1 sequences (189 bp). The authenticity of the sequences is confirmed by sequencing. Plasmids are linearized 5′ to the insert with SpeI and purified using the QIAquick PCR purification kit (Qiagen, Valencia, Calif.). In vitro RNA transcripts are produced overnight using the T7 RNA polymerase as described in the MEGAscript kit (Ambion, Austin, Tex.). DNA is degraded by DNaseI. RNA transcripts are purified by silica binding using the RNeasy mini kit (Qiagen). The integrity and quantity of the RNA transcripts is determined by capillary electrophoreses (Agilent, Santa Clara, Calif.).

### RNA Extraction

Viral RNA is extracted from cell-free 100 μL of plasma using the EasyMaq system (Biomerieux, Marcy l'Etoile, France). In parallel, RNA is extracted from 140 μL of plasma using the viral RNA mini is extracted from T47D cells using the EasyMaq. Total RNA stocks previously isolated from whole blood from breast cancer and control patients are also used for NASBA assays.

### NASBA Amplification

RNA standards are used as calibrators or 5 μL of viral, cellular or total RNA and amplified with the primers cited above using the NASBA protocol currently used in Biomeriux (Marcy l'Etoile, France). Data is plotted in standard curves displaying time to positivity (TTP) values for both the wild-type and in vitro RNA, and against Log₁₀ concentration of the RNA standards. Viral and cellular RNA titers are extrapolated from standard curves.

### Statistical Analysis

Correlations are calculated by the Spearman's correlation coefficient (rho) using the SPSS software. Statistical differences between the mean HERV-K(HML-2) RNA titer is compared using the independent T-test for two study groups and Oneway ANOVA for several groups in the GRAphPad PRISM Version 5.0 platform.

### Example 6

### Endogenous Retroviruses are Present in the Plasma of Patients with Lymphoma

### Materials and Methods

Plasma samples from two different patients with large cell lymphoma were centrifuged at 2300 rpm to remove cellular debris. They were then overlayed onto a 10 to 50% sucrose gradient, and centrifuged at 100,000 g for 16 h at 4° C. One mL fractions were collected, and tested for reverse transcriptase (RT) activity using the Enz Check Reverse Transcriptase assay kit (Invitrogen, Carlsbad, Calif.). As well, HERV-K RNA titers were assessed by Real Time RT-PCR as described above.

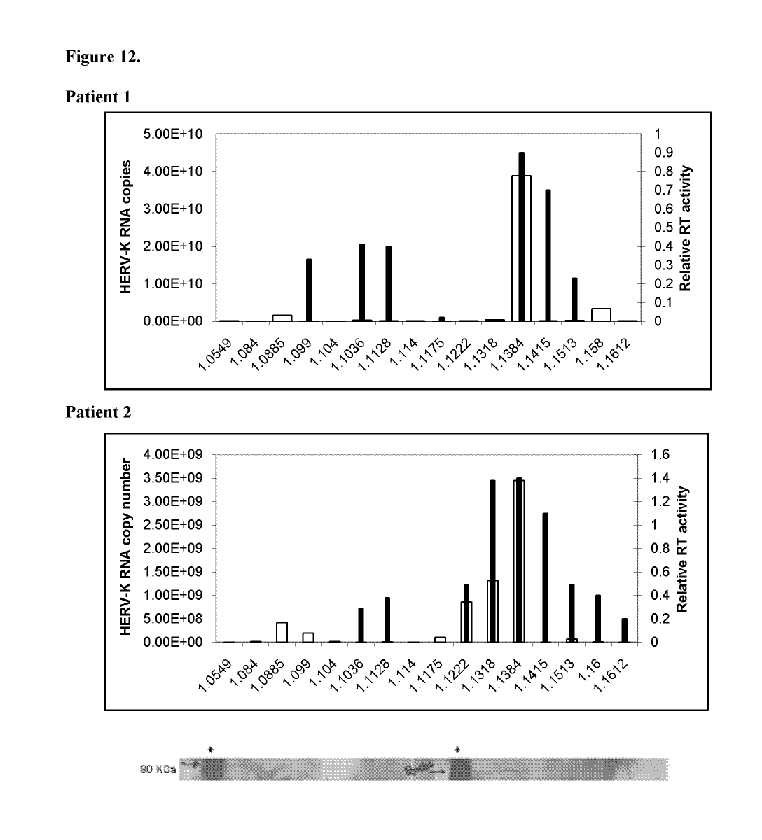

For Western blotting, 1 mL of each fraction was denatured into a final concentration of 2% SDS, and the proteins were extracted by methanol/chloroform precipitation. 20 μg of total protein was loaded in each lane and separated by 10% SDS-PAGE. Proteins were transferred onto nitrocellulose membranes by Western blotting. The blots were immersed in blocking solution and incubated with anti-HERV-K env-specific monoclonal antibody (Herm-1811-5; Austral Biologicals, San Ramon, Calif.). The membranes were washed 5 times, and HERV-K envelope proteins were detected with alkaline-phosphatase-conjugated secondary antibody. As a positive control, lysates from the HERV-K particle-producing NCCIT cell line were used, as identified by two lanes in FIG. 12 with prominent bands at 80 KDa.

### Results

The density of each fraction is given on the X axis of FIG. 12 with data from each of the two patients in the top and bottom bar charts, respectively. As shown in FIG. 12, viral RNA (hollow bars) appears fractions with reverse transcriptase activity (solid bars) in both patients. Western blots show HERV-K envelope protein in the same gradient fractions as RT activity and HERV-K RNA. These data show that endogenous retrovirus is present in the plasma of patients with lymphoma, and that HERV-K RNA, RT activity and HERV-K env proteins band together in sucrose fractions with densities 1.13-1.16 g/mL as expected for retroviral particles.

### Example 7

### Viral Envelope Protein is Present in the Plasma of Large Cell Lymphoma Patients, and Endogenous Retrovirus Circulates in the Blood of Large Cell Lymphoma Patients

### Materials and Methods

Unfractionated plasma samples from three different large cell lymphoma patients with high HERV-K (HML-2) titers in their blood as measured by RT-PCR were resuspended in 10 mL of PBS, and overlayed onto 30% iodoxinol cushions. The pellets were resuspended in PBS, denatured with SDS, and the proteins were extracted by methanol/chloroform precipitation.

### Results

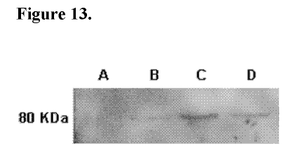

Western blot examinations performed on the patient samples, and on a negative control sample, are shown in FIG. 13. Lane A shows cell lysate of HERV-K particle-negative cell line PA-1. Lanes B, C and D show plasma samples from large cell lymphoma patients with high HERV-K RNA titers. These data show the presence of the viral envelope protein in the plasma of large cell lymphoma patients, and show that endogenous retrovirus circulates in the blood of large cell lymphoma patients.

### Example 8

### Type-1, But not Type-2, HERV-K (HML-2) is Found in the Blood of Patients with Hodgkin's Disease

### Materials and Methods

Using RT-PCR, HERV-K (HML-2) env SU sequences present in the blood of patients with Hodgkin's Disease was characterized. A phylogenetic neighbor-joining (NJ) tree was constructed using the Kimura 2-parameter model. The stability of the branches was evaluated by bootstrap tests with 1000 replications.

### Results

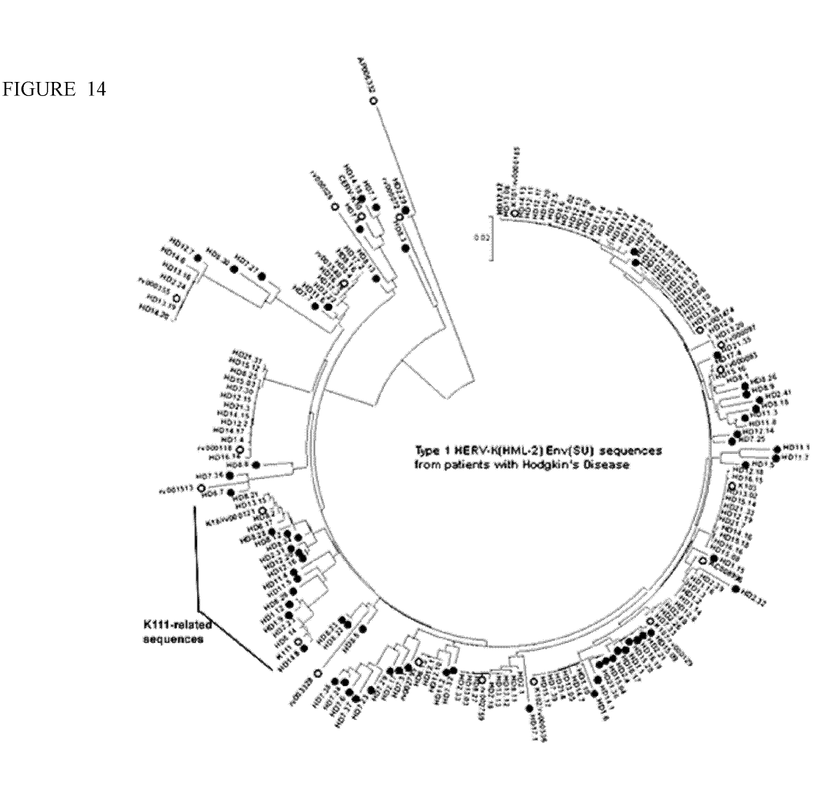

As shown in FIG. 14, the NJ tree is unrooted with taxa arranged for a balanced shape. Hollow circles represent HERV-K proviruses in the HERVd and NCBI databases. Black solid circles represent putative recombinant unresolved taxonomic units (TU)s (less than 95% similar to the parent virus). Clustering of sequences related to a consensus K111 sequence (left) is indicated (K-111-related sequences). The scale bar represents 2% evolutionary distance. Only Type-1, but not Type-2, HERV-K (HML-2) is present in the blood of patients with Hodgkin's Disease, indicating specificity suitable for diagnostic testing. Moreover, the virus shows variation and recombination consistent with active replication.

### Example 9

### Patients with Large Cell Lymphoma have Both Type 1 and Type 2 HERV-K (HML-2) in Plasma, Whereas Viral Sequences from the Blood of Breast Cancer Patients Show Very Little Recombination or Variation

### Materials and Methods

Using RT-PCR, HERV-K (HML-2) env SU sequences present in the blood of patients with large cell lymphoma and breast cancer was characterized. A phylogenetic neighbor-joining (NJ) tree was constructed using the Kimura 2-parameter model. The stability of the branches was evaluated by bootstrap tests with 1000 replications.

Results

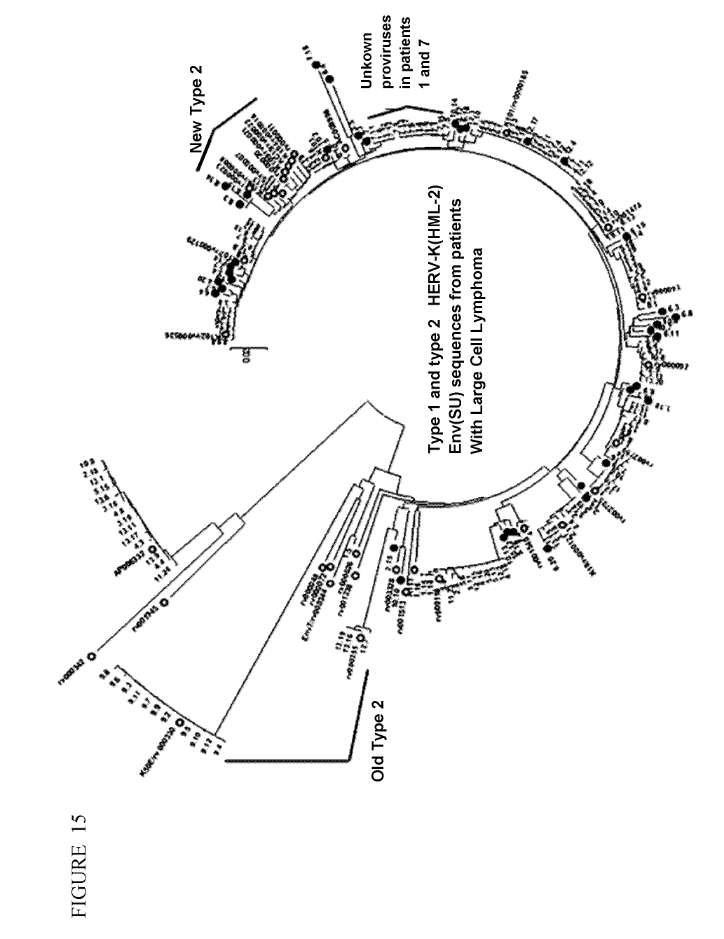

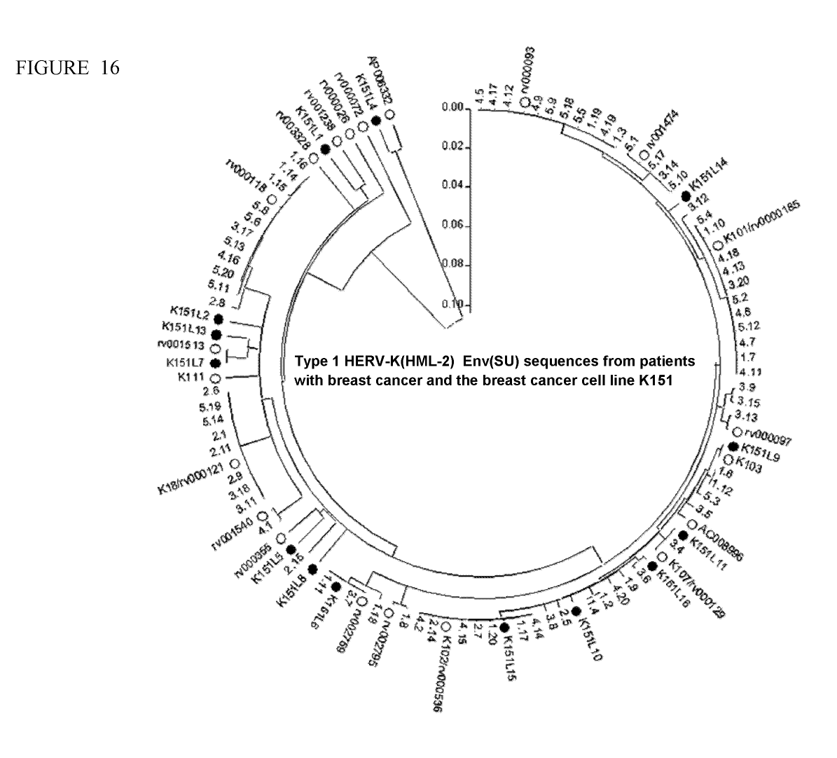

As shown in FIG. 15, the tree from patients with large cell lymphoma is unrooted with taxa arranged for a balanced shape. Hollow circles represent HERV-K proviruses in the HERVd and NCBI databases. Black solid circles represent recombinant unresolved taxonomic units (TU)s (less than 95% similarity to the parent virus). Viruses are Type-1 unless otherwise indicated. The scale bar represents 2% evolutionary distance. The provirus K50E is specifically activated in patient 9. Previously unknown proviral sequences amplified in patients 1 and 7 are indicated at the right. As shown in FIG. 15, patients with large cell lymphoma have both Type 1 and Type 2 HERV-K (HML-2) in their plasma. The presence of recombination is consistent with replication of the virus. As shown in FIG. 16, the tree from patients with breast cancer is unrooted, with taxa arranged for a balanced shape. Hollow circles represent reported HERV-K proviruses in the HERVd and NCBI databases. Solid circles represent putative recombinant unresolved taxonomic units (TU)s (less than 95% similarity to the parent virus) and were only evidenced in the breast cancer cell line K151. The scale bar represents 2% evolutionary distance. No recombinant sequences were observed in breast cancer patients. Viral sequences from the blood of breast cancer patients show very little recombination or variation. Accordingly, the genetic profiles of HERV-K (HML-2) viruses found in the blood of the breast cancer, Hodgkin's Disease, and large cell lymphoma patients are different, indicating disease-specific differential replication of HERV-K (HML-2) of use as a diagnostic modality in cancer, and as a target for therapeutic modalities and vaccines.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of diagnosing cancer in a subject, comprising:
a) providing a sample from a subject;
b) contacting said sample with one or more reagents sufficient for detection of an HERV-K(HML-2) target;
c) measuring an amount of said HERV-K(HML-2) target in said sample; and
d) detecting cancer or the risk of cancer in said subject based on said amount of said HERV-K(HML-2) target in said sample.

2. The method of claim 1, wherein said cancer is selected from a group consisting of an HIV-related cancer and an HIV-unrelated cancer.

3. The method of claim 2, wherein said HIV-related cancer is selected from a group consisting of HIV/AIDS positive large cell lymphoma, HIV/AIDS positive central nervous system lymphoma, HIV positive Hodgkin's disease, and HIV positive T cell leukemia.

4. The method of claim 2, wherein said HIV-unrelated cancer is selected from the group consisting of HIV negative large cell lymphoma, HIV negative Hodgkin's disease, breast cancer and chronic lymphocytic leukemia.

5. The method of claim 1, wherein said HERV-K(HML-2) target is a nucleic acid.

6. The method of claim 5, wherein said HERV-K(HML-2) nucleic acid target is RNA.

7. The method of claim 5, wherein said HERV-K(HML-2) target nucleic acid is selected from the group consisting of gag nucleic acid and env nucleic acid.

8. The method of claim 7, wherein said HERV-K(HML-2) env target nucleic acid corresponds to the diagnosis of a specific HIV-related or HIV-unrelated cancer.

9. The method of claim 1, wherein said measuring said amount of said HERV-K (HML-2) target uses nucleic acid sequence based amplification (NASBA).

10. The method of claim 9, wherein said nucleic acid sequence based amplification (NASBA) comprises use of one or more primers or probes comprising one or more sequences selected from the group consisting of SEQ. ID. NO: 1, SEQ. ID. NO: 2, SEQ. ID. NO: 3, SEQ. ID. NO: 4, SEQ. ID. NO: 5, SEQ. ID. NO: 6, SEQ. ID. NO: 7, SEQ. ID. NO: 8, and SEQ. ID. NO: 9.

11. The method of claim 1, wherein said HERV-K (HML-2) target is HERV-K(HML-2) RNA and said amount of said target is equal to or greater than 103 copies of HERV-K(HML-2) RNA/mL.

12. The method of claim 1, wherein said detecting cancer or the risk of cancer in said subject comprises detecting a response to therapy.

13. The method of claim 12, wherein said detecting is detecting a decrease of HERV-K(HML-2) RNA copies/mL after therapy.

14. The method of claim 12, wherein said HERV-K(HML-2) target is HERV-K(HML-2) RNA and said amount of said target is equal to or less than 103 copies of HERV-K(HML-2) RNA/mL.

15. The method of claim 1, wherein said HERV-K(HML-2) target is a polypeptide.

16. A method for screening compounds, comprising:
a) providing:
i) a sample from a subject suspected of having cancer;
ii) one or more reagents sufficient for the detection of an HERV-K(HML-2) target; and
iii) one or more test compounds;
b) contacting said biological sample with said one or more test compounds; and
c) detecting an amount of said HERV-K(HML-2) target in said sample using said reagents.

17. The method of claim 16, wherein said test compound is selected from the group consisting of a small molecule and an antibody.

18. The method of claim 16, wherein said test compound inhibits the interaction of an HERV-K(HML-2) target with a second compound.

19. A kit for diagnosing cancer in a subject, comprising
a) one or more reagents sufficient for detection of an HERV-K(HML-2) target in a sample; and
b) a computer program on a computer readable medium comprising instructions which direct a processor to analyze data derived from use of said reagents to indicate the presence or absence of cancer in a subject.

20. The kit of claim 19, wherein said one or more reagents sufficient for detection of an HERV-K(HML-2) target are reagents configured for nucleic acid sequence based amplification (NASBA).


SEQUENCE LISTING (24 sequences)

SEQ ID NO: 1 - DNA, 24 bp, Artificial Sequence
  Synthetic
agcaggtcaggtgcctgtaacatt

SEQ ID NO: 2 - DNA, 24 bp, Artificial Sequence
  Synthetic
tggtgccgtaggattaagtctcct

SEQ ID NO: 3 - DNA, 26 bp, Artificial Sequence
  Synthetic
aagacccaaccaccagtagcctatca

SEQ ID NO: 4 - DNA, 23 bp, Artificial Sequence
  Synthetic
agaaaagggcctccacggagatg

SEQ ID NO: 5 - DNA, 20 bp, Artificial Sequence
  Synthetic
ctctccctaggcaaatagga

SEQ ID NO: 6 - DNA, 28 bp, Artificial Sequence
  Synthetic
acggagatggtaacaccagtcacatgga

SEQ ID NO: 7 - DNA, 25 bp, Artificial Sequence
  Synthetic
agacaccgcaatcgagcaccgttga

SEQ ID NO: 8 - DNA, 24 bp, Artificial Sequence
  Synthetic
atcaaggctgcaagcagcatactc

SEQ ID NO: 9 - DNA, 26 bp, Artificial Sequence
  Synthetic
aagttgccatccaccaagaaggcaga

SEQ ID NO: 10 - DNA, 22 bp, Artificial Sequence
  Synthetic
agaaggaaaaggtccagaatta

SEQ ID NO: 11 - DNA, 22 bp, Artificial Sequence
  Synthetic
agacttgtatctggcctcaact

SEQ ID NO: 12 - DNA, 19 bp, Artificial Sequence
  Synthetic
gactataaaggcgaaattc

SEQ ID NO: 13 - DNA, 20 bp, Artificial Sequence
  Synthetic
aggtgagaacgaaggctcaa

SEQ ID NO: 14 - DNA, 26 bp, Artificial Sequence
  Synthetic
tccccttggaatactcctgttttygt

SEQ ID NO: 15 - DNA, 27 bp, Artificial Sequence
  Synthetic
cattccttgtggtaaaactttccaytg

SEQ ID NO: 16 - DNA, 23 bp, Artificial Sequence
  Synthetic
actgcaattaaagtaaaaatgaa

SEQ ID NO: 17 - DNA, 21 bp, Artificial Sequence
  Synthetic
gctgtagcaggagttgcattg

SEQ ID NO: 18 - DNA, 23 bp, Artificial Sequence
  Synthetic
taatcgatgtacttccaatggtc

SEQ ID NO: 19 - DNA, 21 bp, Artificial Sequence
  Synthetic
aaatctctcgtcccaccttac

SEQ ID NO: 20 - DNA, 26 bp, Artificial Sequence
  Synthetic
ttagaacctctcatttcctttccatc

SEQ ID NO: 21 - DNA, 22 bp, Artificial Sequence
  Synthetic
cttgatgtgtagggaagggagg

SEQ ID NO: 22 - DNA, 17 bp, Artificial Sequence
  Synthetic
gcgcggctacagcttca

SEQ ID NO: 23 - DNA, 22 bp, Artificial Sequence
  Synthetic
tctccttaatgtcacgcacgat

SEQ ID NO: 24 - DNA, 23 bp, Artificial Sequence
  Synthetic
ttcatttttactttaattgcagt